(19) United States
(12) Patent Application Publication
(10) Pub. No.: US 2008/0300208 A1
(43) Pub. Date: Dec. 4, 2008

(54) Isociatrate dehydrogenase and uses therof

(75) Inventors: Paz Einat, Nes Zionna (IL); Louis Deiss, Chicago, IL (US); Ruth Maya, Moshav Rinnatia (IL)

Correspondence Address: Cooper & Dunham LLP, 1185 Avenue of the Americas, New York, NY, 10036, US

(73) Assignee: Quark Pharmaceuticals, Inc.

(21) Appl. No.: 12/069,188

(22) Filed: Feb. 7, 2008

Related U.S. Application Data
(63) Continuation of application No. 10/618,143, filed on Jul. 11, 2003, now abandoned.
(60) Provisional application No. 60/395,364, filed on Jul. 11, 2002.
(60) Provisional application No. 60/428,805, filed on Nov. 25, 2002.

(51) Int. Cl. A61K 31/7088; C07H 21/04; C12N 15/85; C12N 5/08; C12N 5/06

(52) U.S. Cl. 514/44; 536/24.5; 435/320.1; 435/375

(57) ABSTRACT

The present invention discloses uses for the IDH gene and/or polypeptide and/or modulators thereof in the diagnosis and treatment of apoptosis-related diseases.

### PRIORITY

This application claims the benefit of U.S. provisional patent application No. 60/395,364, filed 11 Jul. 2002, and of U.S. provisional patent application No. 60/428,805, filed 25 Nov. 2002, which are both hereby incorporated by reference in their entirety.

### FIELD OF THE INVENTION

This invention relates to the field of treatment of apoptosis-related diseases, and screening for novel modulators of such diseases.

### BACKGROUND OF THE INVENTION

Apoptosis, also known as ‘programmed cell death’, is an intrinsic program of cell self-destruction or “suicide”, which is inherent in every eukaryotic cell. In response to a triggering stimulus, cells undergo a highly characteristic cascade of events manifested by cell shrinkage, blebbing of cell membranes, chromatin condensation and fragmentation, culminating in cell conversion to clusters of membrane-bound particles (apoptotic bodies), which are thereafter engulfed by macrophages (Wyllie A H., et al., Int Rev. Cytol 68:251-306, 1980).

Apoptosis is now recognized as one of the more important biological processes, having a major role in normal tissue development and homeostasis. Moreover, derangement of apoptosis control has a role in the pathogenesis of numerous medical disorders, ranging from disorders of excessive apoptosis such as neurodegenerative disorders (e.g., Alzheimer's disease or Parkinson's disease), to disorders wherein death of defective cells is inappropriately inhibited, such as cancer (Bursch, W., et al., Trends Pharmacol. Sci., 13:245-251, 1992).

Tumor drug resistance is a major problem in the treatment of cancer by chemotherapy. In the common epithelial malignancies of adult life—carcinomas of the breast, colon and lung—the impact of chemotherapy has been disappointing. In the last few years, increasing efforts have been invested in obtaining a greater understanding of the response and resistance of cancer cells to chemotherapy by focussing on the role of apoptosis. The rationale behind this approach is that a mechanistic understanding of apoptosis will improve the chances of overcoming tumor drug resistance.

Apoptosis can be thought of as a “default” process, intrinsic to all cells, which is abrogated by the provision of survival signals. A framework for drug-induced apoptosis can be described in which a balance exists between intrinsic and extrinsic survival signals and drug-induced death signals. Pro- and anti-apoptotic signals impact upon apoptotic proteins which ultimately control the apoptotic process. This framework suggests multiple points at which therapeutic interventions could be made to overcome drug resistance and, in addition, generates novel molecular targets for the induction of apoptosis in cancer and other cells. Two areas of fundamental importance are the identification of novel agents, informed by a mechanistic understanding of the process of drug-induced apoptosis, and the modulation of cellular resistance to conventional agents, which would derive from a knowledge of the mechanisms that allow cancer cells to evade apoptosis after drug-induced damage (Makin, G. and Dive, C. Trends in Cell Biology 11:S22-S26, 2001).

### SUMMARY OF THE INVENTION

Applicants have unexpectedly discovered that the IDH1 and IDH2 gene and/or polypeptide products play a role in preventing apoptosis, are anti-apoptopic, and provide a positive viability signal to the FAS induced apoptotic pathway. Furthermore, applicants have discovered that inhibition of expression of the IDH1 or IDH2 gene or neutralization of the expression products promotes cell death.

In accordance with these discoveries, the present invention provides methods for treating apoptosis related diseases, pharmaceutical compositions for treating apoptosis related diseases, diagnostic and prognostic processes in connection with apoptosis relates diseases, and screening processes aimed at obtaining IDH modulators.

### DETAILED DESCRIPTION OF THE INVENTION

In the following description and claims use will be made, at times, of a variety of terms, and the meaning of such terms as they should be construed in accordance with the invention is as follows:

“apoptosis”—a physiological type of cell death which results from activation of some cellular mechanisms, i.e. death which is controlled by the machinery of the cell. Apoptosis may, for example, be the result of activation of the cell machinery by an external trigger, e.g. a cytokine or anti-FAS antibody, which leads to cell death or by an internal signal. The term “programmed cell death” may also be used interchangeably with “apoptosis”.

“apoptosis-related disease”—a disease whose etiology is related either wholly or partially to the process of apoptosis. The disease may be caused either by a malfunction of the apoptotic process (such as in cancer or an autoimmune disease) or by overactivity of the apoptotic process (such as in certain neurodegenerative diseases).

“Cancer” or “Tumor”—an uncontrolled growing mass of abnormal cells. These terms include both primary tumors, which may be benign or malignant, as well as secondary tumors, or metastases which have spread to other sites in the body. Examples of cancer-type diseases include, inter alia: carcinoma (e.g.: breast, colon and lung), leukemia such as B cell leukemia, lymphoma such as B-cell lymphoma, blastoma such as neuroblastoma and melanoma.

The term “polynucleotide” refers to any molecule composed of DNA nucleotides, RNA nucleotides or a combination of both types, i.e. that comprises two or more of the bases guanidine, citosine, timidine, adenine, uracil or inosine, inter alia. A polynucleotide may include natural nucleotides, chemically modified nucleotides and synthetic nucleotides, or chemical analogs thereof. The term encompasses “oligonucleotides” and “nucleic acids”. A polynucleotide generally has from about 75 to 10,000 nucleotides, optionally from about 100 to 3,500 nucleotides. An oligonucleotide refers generally to a chain of nucleotides extending from 2-500 nucleotides.

“Amino acid”—a molecule which consists of any one of the 20 naturally occurring amino acids, amino acids which have been chemically modified (see below), or synthetic amino acids.

“Polypeptide”—a molecule composed of amino acids. The term includes peptides, polypeptides, proteins and peptidomimetics,

A “peptidomimetic” is a compound containing non-peptidic structural elements that is capable of mimicking the biological action(s) of a natural parent peptide. Some of the classical peptide characteristics such as enzymatically scissille peptidic bonds are normally not present in a peptidomimetic.

By “silencing RNA” (siRNA) is meant an RNA molecule which decreases or silences the expression of a gene/mRNA of its endogenous or cellular counterpart. The term is understood to encompass “RNA interference” (RNAi), and “double-stranded RNA” (dsRNA). For recent information on these terms and proposed mechanisms, see Bernstein E., Denli A M., Hannon G J: The rest is silence. RNA. 2001 November; 7(11):1509-21; and Nishikura K.: A short primer on RNAi: RNA-directed RNA polymerase acts as a key catalyst. Cell. 2001 Nov. 16; 107(4):415-8. One example of an siRNA, is the siRNA depicted in FIG. 254, which is a novel siRNA for the IDH gene, and is considered part of the present invention. In one embodiment, the present invention therefore comprises an siRNA molecule for the IDH gene, having the sequence set forth in FIG. 4 (SEQ ID NO:6), and a vector comprising said siRNA.

By the term “antisense” (AS) or “antisense fragment” is meant a nucleic acid fragment having inhibitory antisense activity, said activity causing a decrease in the expression of the endogenous genomic copy of the corresponding gene (in this case IDH). The sequence of the AS is designed to complement a target mRNA of interest and form an RNA:AS duplex. This duplex formation can prevent processing, splicing, transport or translation of the relevant mRNA. Moreover, certain AS nucleotide sequences can elicit cellular RNase H activity when hybridized with their target mRNA, resulting in mRNA degradation (Calabretta et al, 1996: Antisense strategies in the treatment of leukemias. Semin Oncol. 23(1):78-87). In that case, RNase H will cleave the RNA component of the duplex and can potentially release the AS to further hybridize with additional molecules of the target RNA. An additional mode of action results from the interaction of AS with genomic DNA to form a triple helix which can be transcriptionally inactive. The AS fragment of the present invention optionally has the sequence depicted in FIG. 3 or a homologous sequence thereof. Particular AS fragments are the AS of the DNA encoding the particular fragments of IDH described herein.

“Conservative substitution”—refers to the substitution of an amino acid in one class by an amino acid of the same class, where a class is defined by common physicochemical amino acid side chain properties and high substitution frequencies in homologous polypeptides found in nature, as determined, for example, by a standard Dayhoff frequency exchange matrix or BLOSUM matrix. Six general classes of amino acid side chains have been categorized and include: Class I (Cys); Class II (Ser, Thr, Pro, Ala, Gly); Class III (Asn, Asp, Gln, Glu); Class IV (H is, Arg, Lys); Class V (Iie, Leu, Val, Met); and Class VI (Phe, Tyr, Trp). For example, substitution of an Asp for another class III residue such as Asn, Gln, or Glu, is a conservative substitution.

“Non-conservative substitution” —refers to the substitution of an amino acid in one class with an amino acid from another class; for example, substitution of an Ala, a class II residue, with a class III residue such as Asp, Asn, Glu, or Gln.

“Chemically modified”—when referring to the product of the invention, means a product (polypeptide) where at least one of its amino acid residues is modified either by natural processes, such as processing or other post-translational modifications, or by chemical modification techniques which are well known in the art. Among the numerous known modifications typical, but not exclusive examples include: acetylation, acylation, amidation, ADP-ribosylation, glycosylation, GPI anchor formation, covalent attachment of a lipid or lipid derivative, methylation, myristlyation, pegylation, prenylation, phosphorylation, ubiqutination, or any similar process.

“IDH gene”—the isocitrate dehydrogenase 1 coding sequence open reading frame, as shown in FIG. 1 (SEQ ID NO:1), or the isocitrate dehydrogenase 2 coding sequence open reading frame, as shown in FIG. 2 (SEQ ID NO:3), or any homologous sequence thereof preferably having at least 70% identity, more preferable 80% identity, even more preferably 90% or 95% identity. This encompasses any sequences derived from SEQ ID NO:1 or SEQ ID NO:3 which have undergone mutations as described herein.

“IDH polypeptide” refers to the polypeptide of the IDH1 or IDH2 gene, and is understood to include, for the purposes of the instant invention, the terms “oxalosuccinate decarboxylase”, “ICD”, “CID”, “IDP”, “IDPS”, “PICD”, “ICDH”, “HCID”, “IDHM”, “ICD-M”, “mNADP-IDH” and “NADP+-specific IDHH”, derived from any organism, optionally man, splice variants and fragments thereof retaining viability activity, and homologs thereof, preferably having at least 70%, more preferably at least 80%, even more preferably at least 90% or 95% homology thereto. In addition, this term is understood to encompass polypeptides resulting from minor alterations in the IDH1 or IDH2 coding sequence, such as, inter alia, point mutations, substitutions deletions and insertions which may cause a difference in a few amino acids between the resultant polypeptide and the naturally occurring IDH1 or IDH2. Polypeptides encoded by nucleic acid sequences which bind to the IDH1 or IDH2 coding sequence or genomic sequence under conditions of highly stringent hybridization, which are well-known in the art (for example Ausubel et al., Current Protocols in Molecular Biology, John Wiley and Sons, Baltimore, Md. (1988), updated in 1995 and 1998), are also encompassed by this term. Chemically modified IDH1 or IDH2 or chemically modified fragments of IDH1 or IDH2 are also included in the term, so long as the viability activity is retained. The polypeptide sequence of IDH1 is depicted in FIG. 1 (SEQ ID NO: 2). The polypeptide sequence of IDH2 is depicted in FIG. 2 (SEQ ID NO:4).

While mostly IDH1 is exemplified herein, it is to be understood that for all embodiments, IDH2 can effectively replace IDH1, or act in conjunction with or in addition to IDH1.

“Viability activity” defines the capability of the IDH polypeptide to interfere with the apoptotic process in a cell thereby promoting the survival and viability of the cell.

“Biologically active”—the capability of a molecule to modulate the apoptotic process.

“Modulator”—any molecule that is capable of modulation, i.e. that either increases (promotes) or decreases (prevents). The term is understood to include partial or full inhibition, stimulation and enhancement. In the case of a modulator of a polypeptide, such as a the IDH polypeptide, the modulator may be a direct modulator of the biological activity of IDH, or it may be a modulator of the IDH gene; in the latter case, the viability activity of IDH is indirectly modulated by a modulator that affects the transcription or translation of the gene (and does not directly act on the polypeptide). Modulators can include AS fragments, siRNAs, ribozymes, polypeptides, small chemical molecules and pigments, inter alia.

“Inhibitor” generally refers to a molecule which is capable of partially or fully inhibiting the biological activity of a gene or gene product. In the case of IDH, the term refers to a molecule which partially or fully inhibits IDH viability activity. Similarly to a modulator, an inhibitor may be a direct inhibitor of the viability activity of IDH, or it may be an inhibitor of the IDH gene; in the latter case, the viability activity of IDH is indirectly inhibited by an inhibitor that affects the transcription or translation of the gene (and does not directly act on the polypeptide). Examples of different types of inhibitors are, inter alia: nucleic acids such as AS fragments, siRNA, or vectors comprising them; polypeptides such as dominant negatives, antibodies, or, in some cases, enzymes; catalytic RNAs such as ribozymes; small chemical molecules; and pigments. Specific IDH inhibitors are given below.

“Inhibition of apoptosis”-inhibiting or reducing the apoptotic process.

“Having at least X % identity”—with respect to two amino acid or nucleic acid sequence sequences, refers to the percentage of residues that are identical in the two sequences when the sequences are optimally aligned. Thus, 90% amino acid sequence identity means that 90% of the amino acids in two or more optimally aligned polypeptide sequences are identical.

“Expression vector”—refers to vectors that have the ability to incorporate and express heterologous DNA fragments in a foreign cell. Many prokaryotic and eukaryotic expression vectors are known and/or commercially available. Selection of appropriate expression vectors is within the knowledge of those having skill in the art.

“Deletion”—is a change in either nucleotide or amino acid sequence in which one or more nucleotides or amino acid residues, respectively, are absent.

“Insertion” or “addition”—is that change in a nucleotide or amino acid sequence which has resulted in the addition of one or more nucleotides or amino acid residues, respectively, as compared to the naturally occurring sequence.

“Substitution”—replacement of one or more nucleotides or amino acids by different nucleotides or amino acids, respectively. As regards amino acid sequences the substitution may be conservative or non-conservative.

The term “Antibody” refers to IgG, IgM, IgD, IgA, and IgE antibody, inter alia. The definition includes polyclonal antibodies or monoclonal antibodies. This term refers to whole antibodies or fragments of the antibodies comprising the antigen-binding domain of the anti-GPCRV product antibodies, e.g. antibodies without the Fc portion, single chain antibodies, fragments consisting of essentially only the variable, antigen-binding domain of the antibody, etc. The term “antibody” may also refer to antibodies against nucleic acid sequences obtained by cDNA vaccination. The term also encompasses antibody fragments which retain the ability to selectively bind with their antigen or receptor and are exemplified as follows, inter alia:
(1) Fab, the fragment which contains a monovalent antigen-binding fragment of an antibody molecule which can be produced by digestion of whole antibody with the enzyme papain to yield a light chain and a portion of the heavy chain;
(2) (Fab′)₂, the fragment of the antibody that can be obtained by treating whole antibody with the enzyme pepsin without subsequent reduction; F(ab′₂) is a dimer of two Fab fragments held together by two disulfide bonds;
(3) Fv, defined as a genetically engineered fragment containing the variable region of the light chain and the variable region of the heavy chain expressed as two chains; and
(4) Single chain antibody (SCA), defined as a genetically engineered molecule containing the variable region of the light chain and the variable region of the heavy chain linked by a suitable polypeptide linker as a genetically fused single chain molecule.

By the term “epitope” as used in this invention is meant an antigenic determinant on an antigen to which the antibody binds. Epitopic determinants usually consist of chemically active surface groupings of molecules such as amino acids or sugar side chains and usually have specific three dimensional structural characteristics, as well as specific charge characteristics.

The terms “chemical compound”, “small molecule”, “chemical molecule” “small chemical molecule” and “small chemical compound” are used interchangeably herein and are understood to refer to chemical moieties of any particular type which may be synthetically produced or obtained from natural sources and usually have a molecular weight of less than 2000 daltons, less than 1000 daltons or even less than 600 daltons.

“Treating a disease”—refers to administering a therapeutic substance effective to ameliorate symptoms associated with a disease, to lessen the severity or cure the disease, or to prevent the disease from occurring.

“Effective amount”—an amount of a pharmaceutical compound or composition which is effective to achieve an improvement in a patient or his physiological systems including, but not limited to, improved survival rate, more rapid recovery, or improvement or elimination of symptoms, and other indicators as are selected as appropriate determining measures by those skilled in the art.

“in conjunction with”—prior to, simultaneously or subsequent to.

“Detection”—refers to a method of detection of a disease. This term may refer to detection of a predisposition to a disease, or to the detection of the severity of the disease.

“Probe”—the IDH1 or IDH2 polypeptide encoding sequence, a fragment thereof having at least 20-30 nucleotides, or a sequence complementary therewith, when used to detect the presence of other similar sequences in a sample. The detection is carried out by identification of hybridization complexes between the probe and the assayed sequence. The probe may be attached to a solid support or to a detectable label.

### Isocitrate Dehydrogenase (IDH)

Isocitrate dehydrogenases catalyze the oxidative decarboxylation of isocitrate to 2-oxoglutarate (alpha-ketoglutarate), a substrate of the citric acid cycle. These enzymes belong to two distinct subclasses, one of which utilizes NAD(+) as the electron acceptor and the other NADP(+). Five isocitrate dehydrogenases have been reported: three NAD(+)-dependent isocitrate dehydrogenases, which localize to the mitochondrial matrix, and two NADP(+)-dependent isocitrate dehydrogenases, one of which is mitochondrial and the other predominantly cytosolic. Each NADP(+)-dependent isozyme is a homodimer.

Eukaryotic organisms have 3 different IDHs which belong to the same family and have a shared domain of 20 amino acids at the c-terminus, known as the “isocitrate dehydrogenase signature sequence”. The present invention concerns the human cytosolic NADP-dependent IDH; its activity level varies from tissue to tissue, and is regulated during development (Jennings et al., “Cytosolic NADP+-dependent Isocitrate Dehydrogenase”, J. Biol. Chem., 269: 23128-23134, 1994). The regulation of the activity of cytosolic NADP-dependent IDH is influenced by hormones and cellular differentiation, and thus may have a role in proliferative diseases (such as cancer) or disorders of the reproductive system (Jennings & Stevenson, “A study of the control of NADP+-dependent isocitrate dehydrogenase activity during gonadotrophin induced development of the rat ovary”, Eur J Biochem 198: 621-25, 1991). For details on human cytosolic isocitrate dehydrogenase see also U.S. Pat. No. 5,952,177. In addition, the present invention concerns mitochondrial NADP-dependent IDH. The protein encoded by this gene plays a role in intermediary metabolism and energy production. This protein may tightly associate or interact with the pyruvate dehydrogenase complex. For further information on IDH2, see Oh, I. U., et al., “Assignment of the human mitochondrial NADP(+)-specific isocitrate dehydrogenase (IDH2) gene to 15q26.1 by in situ hybridization” Genomics 38 (1), 104-106 (1996).

Particular fragments of the IDH1 polypeptide include amino acids 1-50, 51-100, 101-150, 151-200, 201-250, 251-300, 301-350, 351-400 and 401-414 of the sequence shown in FIG. 1 (SEQ ID NO:2). Further particular fragments of the IDH1 polypeptide include amino acids 25-74, 75-124, 125-174, 175-224, 225-274, 275-324, 325-374 and 375-414 of the sequence shown in FIG. 1 (SEQ ID NO:2). Particular fragments of the IDH2 polypeptide include amino acids 1-50, 51-100, 101-150, 151-200, 201-250, 251-300, 301-350, 351-400 and 401-452 of the sequence shown in FIG. 2 (SEQ ID NO:4). Further particular fragments of the IDH2 polypeptide include amino acids 25-74, 75-124, 125-174, 175-224, 225-274, 275-324, 325-374, 375-424 and 425-452 of the sequence shown in FIG. 2 (SEQ ID NO:4).

Applicants have discovered that the IDH gene and/or its polypeptide product play an important role in preventing apoptosis, i.e. the IDH gene and/or its polypeptide product are anti-apoptopic. Furthermore, applicants have discovered that the inhibition of expression of the IDH gene or neutralization of the expression products promotes cell death.

WO 00/55350 discloses a very long list of nucleic acids encoding polypeptides which are allegedly useful in diagnosing or treating many diseases, e.g. cancer. One of the disclosed polypeptides is the IDH2 polypeptide.

WO 00/055174 discloses many genes that encode so-called “human prostate cancer associated proteins” or “prostate cancer antigens”, which allegedly have a wide array of possible effects such as neuroprotective, cytostatic, cardioactive, immunomodulatory, muscular, vulnerary, gastrointestinal, nephrotropic, antiinfective, gynaecological and antibacterial activities, and can be used in gene therapy. The patent publication further suggests possible uses for prostate cancer antigen polynucleotides for the treatment of disorders such as neural, immune, muscular, reproductive, gastrointestinal, pulmonary, cardiovascular, renal, and proliferative disorders, wounds, and infectious diseases.

WO 99/54461 (corresponds to German patent application 19817948) discloses novel human nucleic acid (cDNA) sequences, that are highly expressed in uterine tumour tissue and which reportedly have anti cancer and cytostatic activity. These sequences can reportedly be used for identification of agents suitable for treatment of uterine or endometrial cancer; or for directly treating these forms of cancer, and for generation of specific antibodies.

U.S. Pat. No. 5,952,177 discloses human cytosolic isocitrate dehydrogenase (HCID), the polynucleotide sequence of which was first identified in a clone from a breast carcinoma cDNA library. The protein has strong sequence homology with the human mitochondrial NADP-dependent isocitrate dehydrogenase (mPICD) protein, and the HCID polynucleotides and polypeptides are reportedly useful for the diagnosis, prevention and treatment of cancer, reproductive disorders, and peroxisome metabolism related disorders.

None of the above publications provide any evidence of the anti-apoptotic activity of NADP+-dependent IDH.

According to one aspect of the invention, to be referred to herein as “the apoptosis-promoting aspect”, agents which inhibit the expression of an IDH gene, or agents which antagonize, inhibit or neutralize the IDH gene products, are used for enabling cells to undergo apoptosis.

Thus the invention provides in this aspect a pharmaceutical composition comprising an inhibitor of a human IDH polypeptide and a pharmaceutically acceptable excipient. The invention further provides a pharmaceutical composition for inducing apoptosis in cells comprising an inhibitor of a human IDH polypeptide and a pharmaceutically acceptable excipient. The invention further provides a pharmaceutical composition for treating an apoptosis-related disease, such as a cancer, comprising an inhibitor of a human IDH polypeptide and a pharmaceutically acceptable excipient. The invention additionally provides a pharmaceutical composition for the potentiation of chemotherapeutic drugs or irradiation in the treatment of an apoptosis-related disease, optionally cancer, comprising an inhibitor of a human IDH polypeptide and a pharmaceutically acceptable excipient. The inhibitor may be, inter alia:

(a) an antisense oligonucleotide complementary to the entire or a portion of a DNA molecule encoding said IDH polypeptide, said oligonucleotide being capable of inhibiting the expression of said polypeptide;
(b) a modified human IDH polypeptide which is capable of inhibiting the viability activity of the unmodified human IDH polypeptide in a dominant negative manner;
(c) an siRNA;
(d) an expression vector comprising a nucleic acid molecule encoding the antisense oligonucleotide of (a), the modified polypeptide of (b) or the siRNA of (c);
(e) an antibody capable of binding a human IDH polypeptide; and
(f) a small chemical molecule.

Examples of IDH inhibitors are, inter alia, bisubstrate inhibitors (e.g., NADP-oxoglutatrate, obtained by covalently linking 2-oxoglutarate with NAD⁺ and NADP⁺, J Enzyme Inhib 2000; 15(3):265-72), O-(carboxymethyl) oxalohydroxamate (Pirrung M C et al., O-Alkyl Hydroxamates as Metaphors of Enzyme-Bound Enolate Intermediates in Hydroxy Acid Dehydrogenases. Inhibitors of Isopropylmalate Dehydrogenase, Isocitrate Dehydrogenase, and Tartrate Dehydrogenase(1) J Org. Chem. 1996 Jul. 12; 61(14):4527-4531), Oxalylglycine (Grissom C B, Cleland W W, Isotope effect studies of the chemical mechanism of pig heart NADP isocitrate dehydrogenase. Biochemistry. 1988 Apr. 19; 27(8):2934-43), 3-bromo-2-ketoglutarate (Ehrlich, R. S., and Colman, R. F., 1987, J. Biol. Chem. 262, 12,614-12,619), beta-mercapto-alpha-ketoglutarate, beta-methylmercapto-alpha-ketoglutarate and beta-methylmercapto-alpha-hydroxyglutarate (Arch Biochem Biophys 1986 Feb. 15; 245(1): 114-24), 2-(4-bromo-2,3-dioxobutylthio)-1,N6-ethenoadenosine 2′,5′-bisphosphate (2-BDB-T epsilon A-2′,5′-DP) (Bailey, J. M., and Colman, R. F., 1987, J. Biol. Chem. 262, 12,620-12,626), aluminum or aluminum ions (Al³⁺) (Biometals 1992 Winter; 5(4):217-21), adriamycin (Minaga T et al., A possible mechanism of adriamycin cardiotoxicity. Inhibition of NADP-linked isocitrate dehydrogenase. Adv Myocardiol. 1983; 4:247-53) and alpha-methylisocitrate (Petcu L G, Plaut G W, NADP-specific isocitrate dehydrogenase in regulation of urea synthesis in rat hepatocytes. Biochem J. 1980 Sep. 15; 190(3):581-92; and Plaut G W, et al., Alpha-methylisocitrate. A selective inhibitor of TPN-linked isocitrate dehydrogenase from bovine heart and rat liver. J Biol. Chem. 1975 Aug. 25; 250(16):63514). Additional examples of IDH inhibitors include the AS fragment depicted in FIG. 3 (SEQ ID NO:5) and the siRNA depicted in FIG. 4 (SEQ ID NO:6).

Further examples of inhibitors include ribozymes or other catalytic small RNAs, and polypeptides having inhibitory activity on the IDH polypeptide or transcription/translation of a polynucleotide encoding the IDH polypeptide.

Applications of the apoptosis-promoting aspect include therapy of diseases or disorders associated with uncontrolled, pathological cell growth, e.g. cancer, psoriasis, autoimmune diseases and others. A particular application is overcoming resistance of cancer cells to chemotherapy due to inhibition of the apoptosis process in these cells. The use of antisense molecules in gene therapy or protein inhibitors, in accordance with the apoptosis-promoting aspect of the invention, may be in conjunction with cytokines, e.g. IFN-γ or TNFα, in the treatment of cytokine-induced apoptosis or in conjunction with other apoptosis activators, e.g. Fas ligand (FasL), or chemotherapeutic agents such as etoposide, 5-FU (5-fluorouracil), cis-platinum, doxorubicin, a vinca alkaloid, vincristine, vinblastine, vinorelbine, taxol, cyclophosphamide, ifosfamide, chlorambucil, busulfan, mechlorethamine, mitomycin, dacarbazine, carboplatinum, thiotepa, daunorubicin, idarubicin, mitoxantrone, bleomycin, esperamicin A1, dactinomycin, plicamycin, carmustine, lomustine, tauromustine, streptozocin, melphalan, dactinomycin, procarbazine, dexamethasone, prednisone, 2-chlorodeoxyadenosine, cytarabine, docetaxel, fludarabine, gemcitabine, herceptin, hydroxyurea, irinotecan, methotrexate, oxaliplatin, rituxin, semustine, tomudex and topotecan, or a chemical analog of one of these chemotherapeutic agents or irradiation such as gamma irradiation.

An additional embodiment of this aspect concerns a method for treating an apoptosis-related disease in a subject comprising administering to said subject a therapeutically effective amount of an inhibitor of a human IDH polypeptide, in a dosage and over a period of time sufficient to inhibit IDH so as to thereby treat the subject. The inhibitor may be an antibody to an IDH polypeptide, a small chemical molecule such as, inter alia, a bisubstrate.inhibitor, NADP oxoglutatrate, 2-(4-bromo-2,3-dioxobutylthio)-1,N6-ethenoadenosine 2′,5′-bisphosphate, o-(carboxymethyl)oxalohydroxamate, oxalylglycine, 3-bromo-2-ketoglutarate, beta-mercapto-alpha-ketoglutarate, beta-methylmercapto-alpha-ketoglutarate, beta-methylmercapto-alpha-hydroxyglutarate, adriamycin and alpha-methylisocitrate; an siRNA molecule, such as the siRNA molecule having the sequence as set forth in FIG. 4 (SEQ ID NO:6); a dominant negative peptide; an antisense fragment such as the AS fragment having the sequence as set forth in FIG. 3 (SEQ ID NO:5); or a vector comprising any of these polynucleotides, as described in (a)-(f) above. Additional examples of IDH inhibitors are given above.

In addition, a method for potentiating a chemotherapeutic treatment of a subject in need thereof is provided, comprising administering to said subject a therapeutically effective amount of an inhibitor of a human IDH polypeptide, according to the options as described above, in a dosage and over a period of time so as to thereby treat the subject.

An additional aspect of the present invention provides for the use of an inhibitor of the human IDH polypeptide in the preparation of a medicament for treatment of an apoptosis-related disease in a subject. The inhibitor may be any of the options disclosed herein, such as an antibody; a small chemical molecule such as a bisubstrate inhibitor, NADP oxoglutatrate, 2-(4-bromo-2,3-dioxobutylthio)-1, N6-ethenoadenosine 2′,5′-bisphosphate, o-(carboxymethyl)oxalohydroxamate, oxalylglycine, 3-bromo-2-ketoglutarate, beta-mercapto-alpha-ketoglutarate, beta-methylmercapto-alpha-ketoglutarate, beta-methylmercapto-alpha-hydroxyglutarate, adriamycin and alpha-methylisocitrate; an siRNA molecule such as the siRNA comprising consecutive nucleotides having the sequence set forth in FIG. 4 (SEQ ID NO:6); a dominant negative peptide; an antisense fragment such as the AS fragment comprising consecutive nucleotides having the sequence set forth in FIG. 3 (SEQ ID NO: 5) or a vector comprising any of these polynucleotides, as described in (a)-(f) above. Further, the apoptosis-related disease may be a cancer.

An additional aspect of the present invention provides for the use of an inhibitor of the human IDH polypeptide in the preparation of a medicament for potentiation of a chemotherapeutic treatment of an apoptosis-related disease in a subject. As described above, the inhibitor may be, inter alia, an antibody to the IDH polypeptide; a small chemical molecule such as a bisubstrate inhibitor, NADP oxoglutatrate, 2-(4-bromo-2,3-dioxobutylthio)-1,N6-ethenoadenosine 2′,5′-bisphosphate, o-(carboxymethyl)oxalohydroxamate, oxalylglycine, 3-bromo-2-ketoglutarate, beta-mercapto-alpha-ketoglutarate, beta-methylmercapto-alpha-ketoglutarate, beta-methylmercapto-alpha-hydroxyglutarate, adriamycin and alpha-methylisocitrate; an siRNA molecule such as the siRNA comprising consecutive nucleotides having the sequence set forth in FIG. 4 (SEQ ID NO:6); a dominant negative peptide; an antisense fragment such as the AS fragment comprising consecutive nucleotides having the sequence set forth in FIG. 3 (SEQ ID NO: 5) or a vector comprising any of these polynucleotides, as described in (a)-(f) above. Further, the apoptosis-related disease may be a cancer.

The inventors have discovered that the IDH gene apparently plays a role in preventing apoptosis, and the inhibition of its expression or neutralization of its expression products promotes cell death. IDH molecules useful in the apoptosis-preventing aspect of the invention may have the nucleic acid sequence of the IDH gene or other sequences which encode a product having a similar biological activity to that of the IDH gene product. Such IDH molecules include polynucleotides having a sequence other than that of the IDH gene but which, owing to the degenerate nature of the genetic code, encode the same protein or polypeptide as that encoded by the IDH gene.

It is well known that it is possible at times to modify a protein by replacing or deleting certain amino acids which are not essential for a certain biological function, or adding amino acids in a region which is not essential for the protein's biological function, without such modification essentially affecting the biological activity of the protein. Thus, an IDH polynucleotide useful in the apoptosis preventing aspect of the invention may also have a modified sequence encoding such a modified protein. The modified sequence has a sequence derived from that of the IDH gene or from that of the above degenerate sequence, in which one or more nucleic acid triplets (in the open reading frame of the sequence), has been added, deleted or replaced, with the polypeptide product encoded thereby retaining the essential biological properties of the IDH product.

Furthermore, it is known that at times, fragments of polypeptides retain the essential biological properties of the parent, unfragmented polypeptide, and accordingly, a IDH polynucleotide useful in the apoptosis preventing aspect of the invention may also have a sequence encoding such fragments. The invention also provides in this aspect an antisense oligonucleotide complementary to the entire or a portion of a DNA molecule encoding said IDH polypeptide, said sequence being capable of inhibiting the expression of said polypeptide. An example of such an antisense oligonucleotide is depicted in FIG. 3.

The invention also provides a modified human IDH polypeptide which is capable of inhibiting the viability activity of the unmodified human IDH polypeptide in a dominant negative manner and is at least 70% homologous thereto. The invention further provides in this aspect an expression vector comprising a DNA molecule encoding the above antisense oligonucleotide or modified polypeptide. One embodiment of the invention is an antibody capable of binding the human IDH polypeptide and partially or fully inactivating the viability activity thereof. One such an antibody is commercially available, and new antibodies may be prepared according to methods known in the art, as described herein. Another embodiment of the invention is a method for the preparation of a pharmaceutical composition for the treatment of an apoptosis-related disease, such as cancer, or for the potentiation of chemotherapeutic drugs in the treatment of an apoptosis-related disease comprising adding a therapeutically effective amount of an inhibitor of the human IDH polypeptide to a pharmaceutically acceptable excipient.

A polynucleotide useful in the apoptosis-promoting aspect of the invention may have a sequence which is an antisense sequence to that of the IDH gene, or an antisense sequence to part of the IDH gene, blocking of which is sufficient to inhibit expression of the IDH gene. The part of the gene to be blocked can be either the coding or the non-coding part of the IDH gene.

A non-limiting example of a specific antisense sequence is an IDH AS fragment, the sequence of which is given in FIG. 3.

A comparison between the polynucleotide coding sequence of the corresponding sense polynucleotide to said IDH AS fragment and the polynucleotide coding sequence of an IDH polypeptide is given in FIG. 5.

Another polynucleotide useful in the apoptosis promoting aspect of the invention is a DNA molecule coding for a modified IDH product which is capable of inhibiting the activities of the unmodified IDH product in a dominant negative manner, such as a catalytically inactive dehydrogenase, or any other modified polypeptide whose presence in the cell interferes with the normal activity of the native polypeptide, for example by producing faulty hetero dimers comprised of modified and unmodified polypeptides which are inactive and the like.

According to another aspect of the present invention, to be referred to herein as “the apoptosis-preventing aspect”, the above IDH DNA molecules, expression vectors comprising them, or IDH polypeptide products are used for promoting the viability and survival of cells in which the apoptosis process is overactive.

Thus the invention provides in this aspect a pharmaceutical composition comprising the human IDH polypeptide, or a fragment thereof having viability activity, and a pharmaceutically acceptable excipient, a pharmaceutical composition for inhibiting apoptosis in a cell comprising the human IDH polypeptide and a pharmaceutically acceptable excipient and a pharmaceutical composition for treating an apoptosis-related disease comprising the human IDH polypeptide and a pharmaceutically acceptable excipient. The invention further provides in this aspect a pharmaceutical composition comprising an expression vector comprising a polynucleotide encoding the human IDH polypeptide or a fragment thereof having viability activity, and a pharmaceutically acceptable excipient and a pharmaceutical composition for inhibiting apoptosis in a cell comprising an expression vector comprising a polynucleotide encoding the human IDH polypeptide and a pharmaceutically acceptable excipient and a pharmaceutical composition for treating an apoptosis-related cell degenerative disease comprising an expression vector which comprises a polynucleotide encoding the human IDH polypeptide and a pharmaceutically acceptable excipient. The invention further provides in this aspect a method for treatment of an apoptosis-related cell degenerative disease in a subject comprising administering to said subject a therapeutically effective amount of the human IDH polypeptide or a therapeutically effective amount of an expression vector comprising a polynucleotide encoding the human IDH polypeptide. The invention also provides a method for the preparation of a pharmaceutical composition comprising adding a therapeutically effective amount of the human IDH polypeptide to a pharmaceutically acceptable excipient. The invention additionally provides a method for the preparation of a pharmaceutical composition comprising adding a therapeutically effective amount of an expression vector comprising a polynucleotide encoding the human IDH polypeptide or a fragment thereof having viability activity, to a pharmaceutically acceptable excipient.

Examples of possible applications of the apoptosis-preventing aspect of the invention are in prevention of cell death in various degenerative neurological diseases, such as Alzheimer's disease or Parkinson's disease, which are associated with premature death of particular subsets of neurons; prevention of death of T-cells in AIDS patients, which death resembles apoptosis; prevention of rejection-associated cell death in transplants which is believed to result, at least in part, from apoptosis; protection of normal cells from the cytotoxic effects of certain anti-cancer therapies; etc. Additional neurodegenerative diseases in which it can be beneficial to inhibit apoptosis, in this case through IDH, include stroke, epilepsy, depression, ALS (Amyotrophic lateral sclerosis), Huntington's disease and any other disease-induced dementia (such as HIV induced dementia for example); and possibly conditions as hypertension, hypertensive cerebral vascular disease, rupture of an aneurysm, a constriction or obstruction of a blood vessel—as occurs in the case of a thrombus or embolus, angioma, blood dyscrasias, any form of compromised cardiac function including systemic hypotension, cardiac arrest or failure, cardiogenic shock, septic shock, spinal cord trauma, head trauma, seizure, bleeding from a tumor.

The invention also provides a method for treatment of an apoptosis-related disease in a subject, preferably a cancer comprising administering to said subject a therapeutically effective amount of an inhibitor of the human IDH polypeptide. The invention further provides a method for potentiating a chemotherapeutic treatment of an apoptosis-related disease, preferably a cancer-type disease, in a subject comprising administering to said subject a therapeutically effective amount of an inhibitor of the human IDH polypeptide in conjunction with a chemotherapeutic agent. The inhibitor in these methods may be, inter alia, one of the inhibitors (a) through (f) as described above.

The apoptosis-promoting and apoptosis-preventing aspects of the invention may employ, for example, gene therapy. “Gene therapy” means gene supplementation where an additional reference copy of a gene of interest is inserted into a patient's cells. As a result, the polypeptide encoded by the reference gene corrects the defect and permits the cells to function normally, thus alleviating disease symptoms. In the present invention, the reference copy is the IDH gene and other polynucleotides of the invention which encode the IDH polypeptide or similar polypeptides having IDH polypeptide viability activity, and the disease is preferably a degenerative disease, most preferably a neurodegenerative disease. Additionally, the use of antisense molecules in gene therapy may be used in accordance with the apoptosis-promoting aspect of the invention.

Gene therapy of the present invention can be carried out in vivo or ex vivo. Ex vivo gene therapy requires the isolation and purification of patient cells, the introduction of a therapeutic gene and the introduction of the genetically altered cells back into the patient. A replication-deficient virus such as a modified retrovirus can be used to introduce the therapeutic IDH gene into such cells. For example, mouse Moloney leukemia virus (MMLV) is a well-known vector in clinical gene therapy trials. See, e.g., Boris-Lauerie et al., Curr. Opin. Genet. Dev., 3, 102-109 (1993).

In contrast, in vivo gene therapy does not require isolation and purification of a patient's cells. The therapeutic gene is typically “packaged” for administration to a patient such as in liposomes or in a replication-deficient virus such as adenovirus as described by Berkner, K. L., in Curr. Top. Microbiol. Immunol., 158, 39-66 (1992) or adeno-associated virus (AAV) vectors as described by Muzyczka, N., in Curr. Top. Microbiol. Immunol., 158, 97-129 (1992) and U.S. Pat. No. 5,252,479. Another approach is administration of “naked DNA” in which the therapeutic gene is directly injected into the bloodstream or muscle tissue. Still another approach is administration of “naked DNA” in which the therapeutic gene is introduced into the target tissue by microparticle bombardment using gold particles coated with the DNA. Gene therapy vectors can be delivered to a subject by, for example, intravenous injection, local administration (see U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

Cell types useful for gene therapy of the present invention include lymphocytes, hepatocytes, myoblasts, fibroblasts, and any cell of the eye such as retinal cells, epithelial and endothelial cells. Preferably the cells are T lymphocytes drawn from the patient to be treated, hepatocytes, any cell of the eye or respiratory or pulmonary epithelial cells. Transfection of pulmonary epithelial cells can occur via inhalation of a neubulized preparation of DNA vectors in liposomes, DNA-protein complexes or replication-deficient adenoviruses. See, e.g., U.S. Pat. No. 5,240,846. For a review of the subject of gene therapy, in general, see the text “Gene Therapy” (Advances in Pharmacology 40, Academic Press, 1997).

The present invention additionally provides a method of preparing a pharmaceutical composition which comprises the steps of: obtaining a compound by any of the methods of the invention and admixing said compound with a pharmaceutically acceptable excipient. This invention also provides a pharmaceutical composition for modulating apoptosis in cells comprising a compound identified by any of the methods of the invention, or a chemical analog or homolog thereof, and a pharmaceutically acceptable excipient.

By “chemical analog” or “chemical homolog” as used herein is meant a molecule derived from the originally identified agent (which may be identified through any of the methods described herein), that retains the activity observed in the parent molecule; chemical analogs or homologs may also share structural properties with the parent inhibitor molecule.

According to a third aspect of the present invention, referred to herein at times as “the screening aspect”, expression of IDH polynucleotides and activity of IDH polypeptides are used in the screening of various compounds in order to obtain those which may be active in modulating the apoptotic process.

In a cell-based embodiment of this aspect of the invention, there is provided a process for obtaining a compound which modulates apoptosis in a cell comprising:

a) providing cells which express the human IDH polypeptide;
b) contacting said cells with said compound; and
c) determining the ability of said compound to modulate apoptosis in the cells.

In a preferred embodiment, the process comprises:

a) providing test cells and control cells which express the human IDH polypeptide at a level at which approximately 50% of the cells undergo apoptosis in the presence of an apoptosis-stimulating agent;
b) contacting said test cells with said compound;
c) treating said cells in conjunction with step (b) with an amount of apoptosis-stimulating agent capable of causing apoptosis in the control cell; and
d) determining the ability of said compound to modulate apoptosis in the test cell.

In another preferred embodiment, the process comprises:

a) providing a test cell which expresses the human IDH polypeptide and a control cell which does not express the human IDH polypeptide;
b) contacting said cells with said compound;
c) treating said cells in conjunction with step (b) with an amount of apoptosis-stimulating agent capable of causing apoptosis in the control cell but not in the test cell in the absence of said compound; and
d) determining the ability of said compound to promote apoptosis in the test cell.

In some methods and processes of the invention, a preferred apoptosis-stimulating agent may be a Fas activating agent such as a Fas ligand or an anti-Fas activating antibody or a chemotherapeutic drug such as those described above, or an analog of one of these chemotherapeutic drugs or a chemical analog or homolog thereof, or irradiation such as gamma irradiation.

It will be appreciated that, based on knowledge of the IDH polypeptide, it is possible to devise a non cell-based assay for screening for, i.e. obtaining, compounds which modulate apoptosis through the human IDH polypeptide. An example of such a non cell-based assay is described in Example IV. Without being bound by theory, the anti-apoptotic effect of the IDH polypeptide may be due to the specific binding or interaction of part or all of the IDH polypeptide to a different species such as, without limitation, a factor, molecule, or specific binding substance, and this effect may be monitored by linking this specific binding or interaction to a signaling system. Some of the screening systems of the present invention can therefore be aimed at obtaining compounds which, for example, modulate or disturb this specific interaction of the IDH polypeptide with such species.

Therefore, in a non cell-based embodiment there is provided a process for obtaining a compound which modulates apoptosis through the human IDH polypeptide comprising:

a) measuring the activity of the human IDH polypeptide, or a fragment thereof having viability activity,
b) contacting said polypeptide or fragment with said compound; and
c) determining whether the activity of said polypeptide or fragment is affected by said compound.

Another non cell-based embodiment provides a process for obtaining a compound which modulates apoptosis through the human IDH polypeptide comprising:

a) measuring the binding of the human IDH polypeptide, or a fragment thereof having viability activity, to a species to which the human IDH polypeptide interacts specifically in vivo to produce an anti-apoptotic effect;
b) contacting said polypeptide or fragment with said compound; and
c) determining whether the activity of said polypeptide or fragment is affected by said compound.

One example of a species to which the human IDH polypeptide interacts specifically in vivo is isocitrate.

Additionally, a kit is provided for obtaining a compound which modulates apoptosis in a cell comprising:

(a) the human IDH polypeptide, or a fragment thereof having viability activity;
(b) a species to which the human IDH polypeptide interacts specifically in vivo to produce an anti-apoptotic effect;
(c) means for measuring the interaction of the human IDH polypeptide, or a fragment thereof having viability activity, to the species;

Additionally, a nucleic acid probe is provided which is capable of hybridizing to at least 20, preferably to at least 30 nucleotides of a DNA polynucleotide encoding the IDH polypeptide.

According to a fourth aspect of the present invention, referred to herein at times as “the diagnostic aspect”, individuals suffering from a disease are examined in order to determine whether the disease is related to the defective activity of the IDH gene and which therapeutic modalities might be effective. Thus the invention provides in this aspect a process for determining the susceptibility of a subject to a chemotherapeutic treatment of an apoptosis-related disease comprising:
(a) providing the average, normal level of the IDH polypeptide in the cells of healthy subjects;
(b) determining the level of the IDH polypeptide in said subject;
(c) comparing the levels obtained in (a) and (b) above, a low level of IDH polypeptide in said subject as compared to the level in healthy subjects indicating a susceptibility of said subject to a chemotherapeutic treatment of said apoptosis-related disease.

The invention additionally provides in this aspect a process for determining the susceptibility of a subject to a chemotherapeutic treatment of an apoptosis-related disease comprising:
(a) providing the average, normal level of mRNA encoding the IDH polypeptide in the cells of healthy subjects;
(b) determining the level of mRNA encoding the IDH polypeptide in said subject;
(c) comparing the levels obtained in (a) and (b) above; a low level of mRNA encoding IDH in said subject as compared to the level in healthy subjects indicating a susceptibility of said subject to a chemotherapeutic treatment of said apoptosis-related disease.

For example, IDH negative cells may be more susceptible to control by chemotherapeutic drugs that work by inducing apoptosis, so that the choice of treatment modalities may be made based on the IDH state of the cells. It is also possible that a high level of IDH gene expression as compared to a control may be used as a marker for tumor cells at a certain stage of cancer development.

In the case of several diseases, including cancer, often the symptoms are the result of the very late stages of disease and thus it would be beneficial to have markers that could diagnose cancer earlier as a result of screening of the general population.

In accordance with this aspect, the examination is carried out by comparing the level of the IDH DNA or polypeptide molecules in a healthy population to the respective level in the individual, or by following RNA and/or protein expression in an individual.

Thus, in this aspect the present invention provides a process of diagnosing a cancer in a subject comprising:
(a) providing the average, normal level of the IDH polypeptide in the cells of healthy subjects;
(b) determining the level of the polypeptide in said subject;
(c) comparing the levels obtained in (a) and (b) above, wherein a high level of the IDH polypeptide in said subject as compared to the level in healthy subjects is indicative of a cancer.

The process may in addition be performed by examining the level of a polynucleotide encoding the IDH polypeptide.

For example, the presence and/or level of IDH DNA molecules may be assessed by Southern blot analysis and/or PCR. The mRNA may be analyzed on Northern blots and/or by reverse-transcription PCR (RT-PCR), followed by sequence analysis and/or by in-situ hybridizations of tissue sections. Protein expression may be monitored in cell extracts by Western analysis, or by in-situ immuno-staining of tissue sections using antibodies to IDH polypeptides. The absence of a IDH gene, a partial deletion or any other difference in the sequence that indicates a mutation in an essential region, or the lack of a IDH RNA and/or polypeptide which may result in a loss of function may indicate that the individual may be treated by chemotherapy without drug resistance due to the IDH polypeptide.

More specifically, measurement of the level of the IDH polypeptide is determined by a method selected from the group consisting of immunohistochemistry (Microscopy, Immunohistochemistry and Antigen Retrieval Methods: For Light and Electron Microscopy, M. A. Hayat (Author), Kluwer Academic Publishers, 2002; Brown C.: “Antigen retrieval methods for immunohistochemistry”, Toxicol Pathol 1998; 26(6): 830-1), western blotting (Laemmeli UK: “Cleavage of structural proteins during the assembley of the head of a bacteriophage T4”, Nature 1970; 227: 680-685; Egger & Bienz, “Protein (western) blotting”, Mol Biotechnol 1994; 1(3): 289-305), ELISA (Onorato et al., “Immunohistochemical and ELISA assays for biomarkers of oxidative stress in aging and disease”, Ann NY Acad Sci 1998 20; 854: 277-90), antibody microarray hybridization (Huang, “detection of multiple proteins in an antibody-based protein microarray system, Immunol Methods 2001 1; 255 (1-2): 1-13) and targeted molecular imaging (Thomas, Targeted Molecular Imaging in Oncology, Kim et al (Eds)., Springer Verlag, 2001).

Measurement of the level of an IDH polynucleotide is determined by a method selected from: RT-PCR analysis, in-situ hybridization (“Introduction to Fluorescence In Situ Hybridization: Principles and Clinical Applications”, Andreeff & Pinkel (Editors), John Wiley & Sons Inc., 1999), polynucleotide microarray and Northern blotting (Trayhurn, “Northern blotting”, Proc Nutr Soc 1996; 55(1B): 583-9; Shifman & Stein, “A reliable and sensitive method for non-radioactive Northern blot analysis of nerve growth factor mRNA from brain tissues”, Journal of Neuroscience Methods 1995; 59: 205-208)

In accordance with this aspect of the present invention, it will be appreciated that the IDH polypeptide and/or polynucleotide may serve as a biomarker for measuring the response of tumors to treatment; monitoring IDH levels in a patient undergoing cancer treatment could then be aimed at following the response of tumors (or other proliferative diseases) to therapy, which allows tailoring of the treatment to the specific needs of the patient.

Therefore, this embodiment of the present invention provides a process for determining the efficacy of a chemotherapeutic treatment administered to a subject comprising:
(a) determining the level of the IDH polypeptide in the subject prior to a treatment;
(b) determining the level of the IDH polypeptide in the subject after the treatment;
(c) comparing the levels obtained in (a) and (b) above, a high level of IDH polypeptide prior to the treatment as compared to the level after the treatment indicating efficacy of the treatment.

Further, the level of the IDH mRNA may be measured in place of the IDH polypeptide, to the same end.

Furthermore, the invention comprehends isolated and/or purified polynucleotides (nucleic acid molecules) and isolated and/or purified polypeptides having at least about 70%, preferably at least about 75%; more preferably at least about 80%, even more preferably at least about 90%, most preferably at least about 95% homology to the IDH polynucleotides and polypeptides disclosed herein. The invention also comprehends that these homologous polynucleotides and polypeptides can be used in the same fashion as the herein or aforementioned polynucleotides and polypeptides.

Nucleotide sequence homology can be determined using the “Align” program of Myers and Miller, ((1988) CABIOS 4:11-17) and available at NCBI. Alternatively or additionally, the term “homology” for instance, with respect to a nucleotide or amino acid sequence, can indicate a quantitative measure of homology between two sequences. The percent sequence homology can be calculated as (Nref−Ndif)*100/Nref, wherein Ndif is the total number of non-identical residues in the two sequences when aligned and wherein Nref is the number of residues in one of the sequences. Hence, the DNA sequence AGTCAGTC has a sequence similarity of 75% to AATCMTC (Nref=8; Ndif=2).

Alternatively or additionally, “homology” with respect to sequences can refer to the number of positions with identical nucleotides or amino acid residues divided by the number of nucleotides or amino acid residues in the shorter of the two sequences wherein alignment of the two sequences can be determined in accordance with the Wilbur and Lipman algorithm ((1983) Proc. is Natl. Acad. Sci. USA 80:726), for instance, using a window size of 20 nucleotides, a word length of 4 nucleotides, and a gap penalty of 4, and computer-assisted analysis and interpretation of the sequence data including alignment can be conveniently performed using commercially available programs (e.g., Intelligenetics™ Suite, Intelligenetics Inc., CA). When RNA sequences are said to be similar, or to have a degree of sequence identity or homology with DNA sequences, thymidine (T) in the DNA sequence is considered equal to uracil (U) in the RNA sequence. RNA sequences within the scope of the invention can be derived from DNA sequences or their complements, by substituting thymidine (T) in the DNA sequence with uracil (U).

Additionally or alternatively, amino acid sequence similarity or homology can be determined, for instance, using the BlastP program (Altschul et al. Nucl. Acids Res. 25:3389-3402) and available at NCBI. The following references provide algorithms for comparing the relative identity or homology of amino acid residues of two polypeptides, and additionally, or alternatively, with respect to the foregoing, the teachings in these references can be used for determining percent homology: Smith et al. (1981) Adv. Appl. Math. 2:482-489; Smith et al. (1983) Nucl. Acids Res. 11:2205-2220; Devereux et al. (1984) Nucl. Acids Res. 12:387-395; Feng et al. (1987) J. Molec. Evol. 25:351-360; Higgins et al. (1989) CABIOS 5:151-153; and Thompson et al. (1994) Nucl. Acids Res. 22:4673-480.

Polynucleotide sequences that are complementary to any of the sequences or fragments encompassed by the present invention discussed above are also considered to be part of the present invention. Whenever any of the sequences discussed above are produced in a cell, the complementary sequence is concomitantly produced and, thus, the complementary sequence can also be used as a probe for the same diagnostic purposes.

The invention has been described in an illustrative manner, and it is to be understood that the terminology which has been used is intended to be in the nature of words of description rather than of limitation.

Throughout this application, various publications, patents and patent applications, including United States patents/applications, are referenced by author and year and patents by number. The disclosures of these publications and patents and patent applications in their entireties are hereby incorporated by reference into this application in order to more fully describe the state of the art to which this invention pertains.

Obviously, many modifications and variations of the present invention are possible in light of the above teachings. It is, therefore, to be understood that within the scope of the appended claims, the invention can be practiced otherwise than as specifically described.

### BRIEF DESCRIPTION OF THE FIGURES

In order to understand the invention and to see how it may be carried out in practice, preferred embodiments will now be described, by way of non-limiting examples only, with reference to the accompanying drawings, in which:

FIG. 1 shows the polynucleotide coding sequence of the IDH1 gene product (SEQ ID NO: 1), and corresponding amino acid sequence (SEQ ID NO:2);

FIG. 2 shows the polynucleotide coding sequence of the IDH2 gene product (SEQ ID NO:3), and corresponding amino acid sequence (SEQ ID NO:4);

FIG. 3 shows the nucleotide sequence of the IDH antisense fragment (SEQ ID NO:5);

FIG. 4 shows the sequence of an IDH siRNA (SEQ ID NO:6);

FIG. 5 is a comparison between the polynucleotide coding sequence of the corresponding sense polynucleotide to the IDH antisense fragment and the polynucleotide coding sequence of the IDH gene;

FIG. 6 is a comparison between the amino acid sequences of the IDH1 and IDH2 polypeptides, i.e., a comparison between SEQ ID NO:2 and SEQ ID NO:4 (amino acids 10-405 and 50-445 respectively);

FIG. 7 and FIG. 8 are graphs illustrating the results of loss of function validation experiments.

FIG. 9 (A and B) are graphs illustrating the results of gain of function validation experiments.

FIG. 10 is a graph which presents the results of an additional loss of function validation experiment.

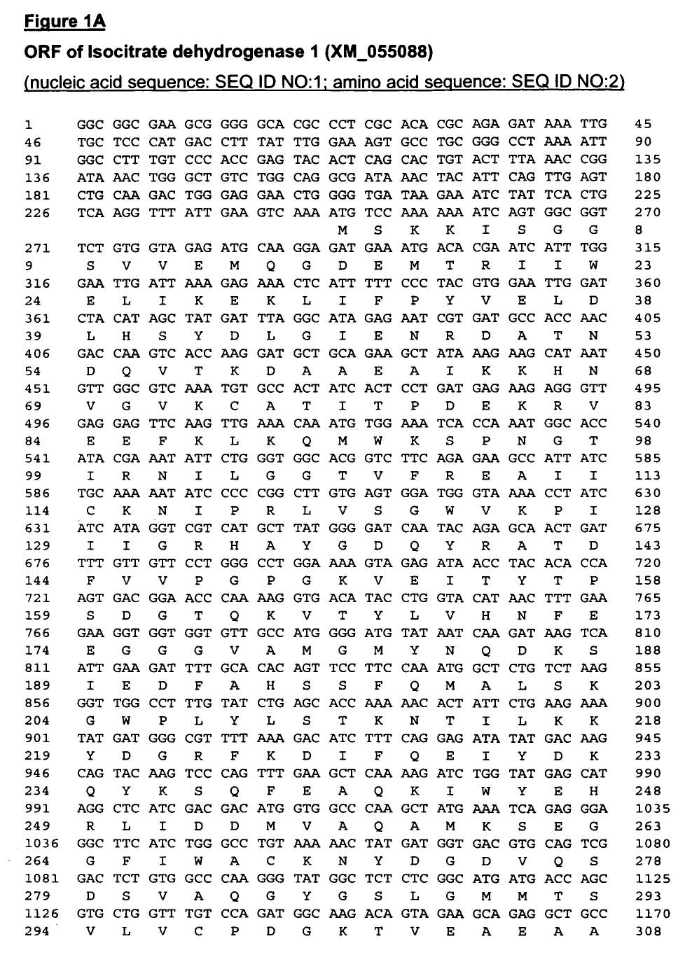

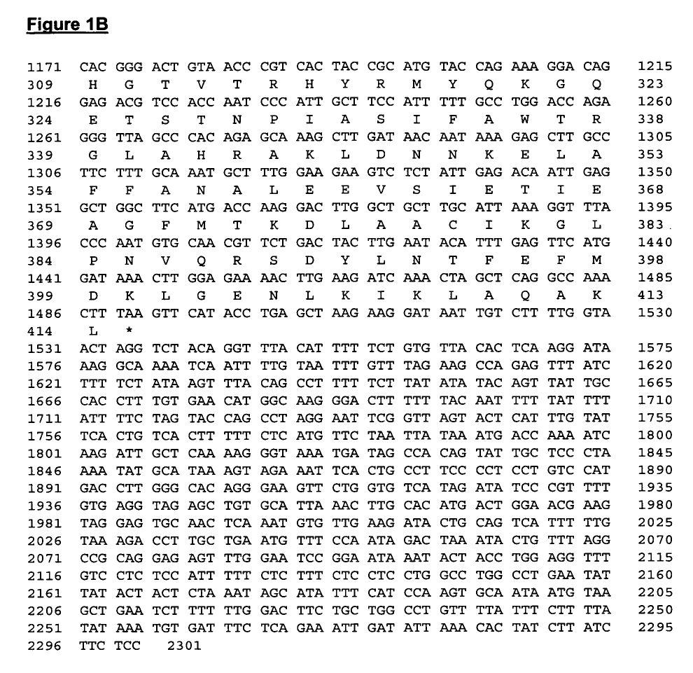

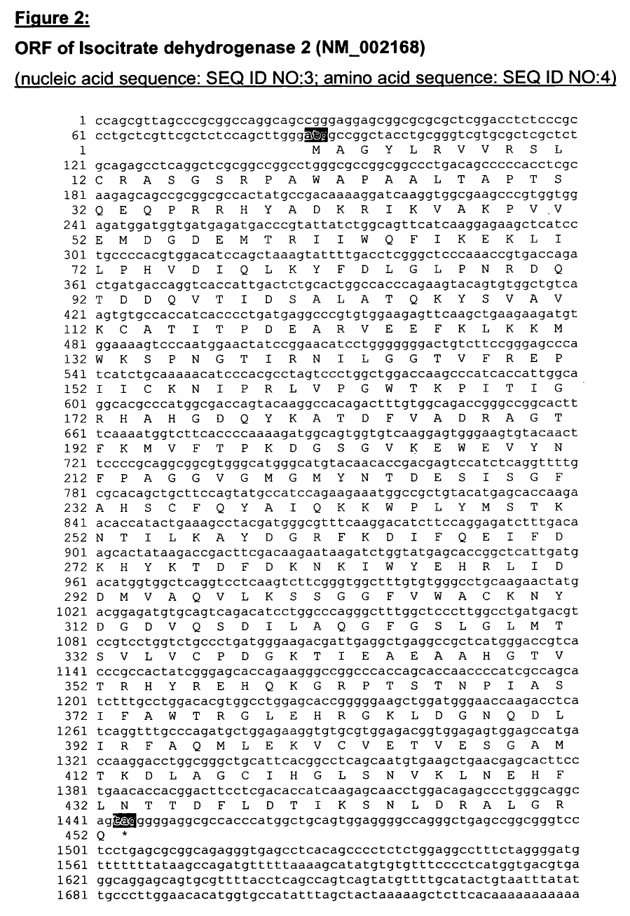

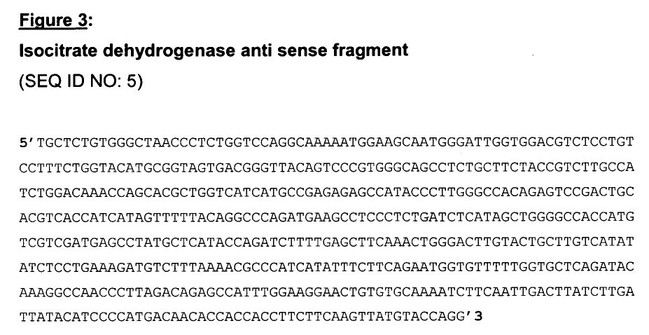

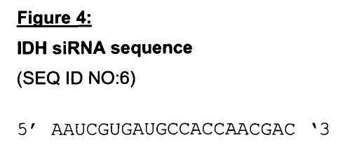

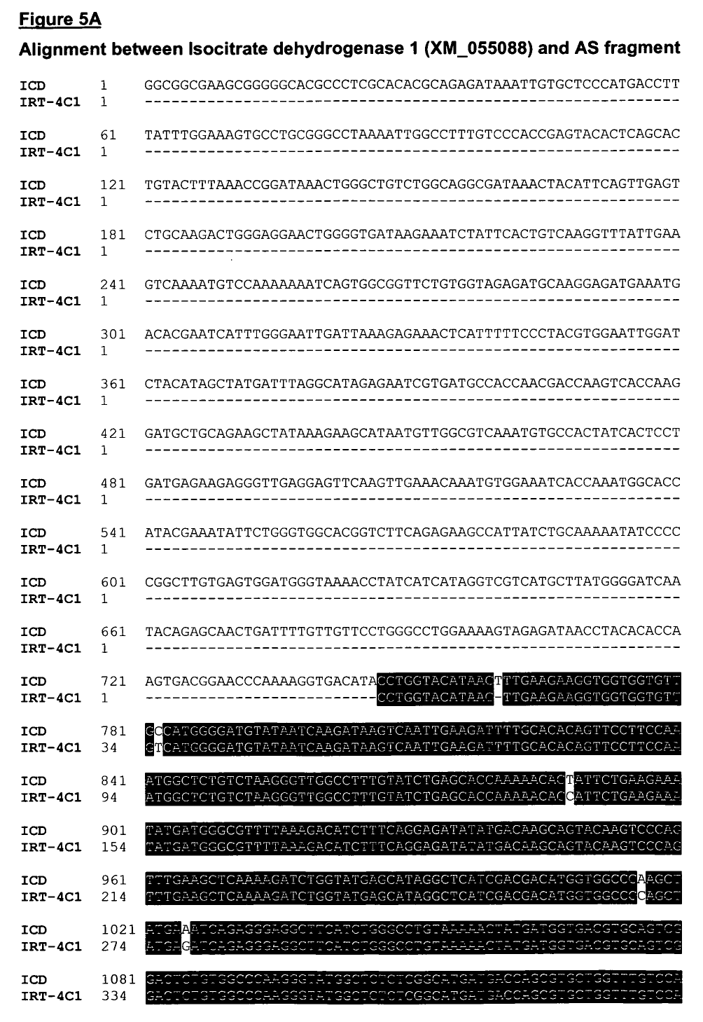

### EXAMPLES

### Example I

### Identification of IDH Gene Fragment

The assignees of the present invention has developed a high throughput method that allows rapid identification of potential anti-cancer targets, and this method has been applied to the identification of genes whose products modulate the apoptotic process. These genes encode proteins that may be targets for the development of anti-cancer therapeutics. Briefly, target genes that are required for tumor cell survival are identified and validated in a cell culture model using a genetic screen termed the Achilles Heel Method (AHM). Acceleration of FAS induced apoptosis, for example, may ameliorate auto-immunity and enhanced tumor suppression. Thus, pharmacological inhibition of the FAS pathway inhibitors can be translated into significant clinical benefits as they will accelerate killing of tumor cells.

In order to identify anti apoptotic genes, HeLa cells were transfected with vectors harboring inactivating cDNA fragments (anti-sense or dominant negative sense fragments) and treated with a sub-optimal dose of apoptotic pathway inducer. Cells harboring inactivated apoptotic inhibitors were more sensitive to apoptosis and thus preferentially killed. The inactivating cDNA fragments contained in the lost cells were identified by hybridization to either a cDNA or an oligonucleotide microarray. Negative differentials between the total, untreated population and the treated population represents the depleted cDNA fragments, expressed in the sensitive cells.

The method of subtraction analysis used to identify the IDH gene as affecting apoptosis is performed essentially as described in U.S. Pat. No. 6,057,111, the entire contents of which are incorporated by reference.

Briefly, the method was applied to HeLa cells treated with activating anti-Fas antibody in order to identify genes that, when knocked-out, cause sensitization of HeLa cells to the action of anti-Fas antibodies. HeLa cells are derived from a human cervical carcinoma and were used in the original TKO selection method (Deiss and Kimchi, Science 252:117-120, 1991). HeLa cells were used as an exemplar of the method of the present system as they are easily grown in culture, are easily transfected and respond to anti-Fas antibody treatment. Anti-Fas antibody (Kamiya Biomedical Company, Seattle, Wash., catalog number: MC-060) is directed against Fas/CD95/Apo-1, a transmembrane receptor that is known to signal a death response in a variety of cell types. This antibody is an activating antibody, that is, the binding of the antibody mimics the effects of binding of ligand. Applying the appropriate dose to responding cells has been shown to lead to induction of cell death (Deiss et al., EMBO Journal 15:3861-3870, 1996). HeLa cells respond to this treatment.

In this example, genes are identified that regulate the sensitivity of HeLa cells to killing by anti-Fas antibody. Specifically, genes are identified whose loss sensitizes HeLa cells to anti-Fas treatment. The outline of the procedure is as follows:

1. HeLa cells were transfected with a fragmented cDNA library enriched for anti-sense fragments.

2. Cells containing anti-sense expression vectors were isolated by selection with Hygromycin B. Since the vector contains the Hygromycin B resistance gene, the selection of the transfected cultures with Hygromycin B generated a population of cells which contain the fragment expression cassettes.

3. Aliquots of this pool of cells were treated with anti-Fas antibody. It should be noted that more than one condition could be screened at the same time. Treatment with a sub-lethal dose of anti-Fas antibody (10 ng/ml) was performed.

Cells that are super-sensitive to treatment with anti-Fas antibody were killed whereas about 50% of the population which is resistant to the treatment proliferated.

4. Aliquots of the cells just before the treatment with anti-Fas antibody and just after the treatment with anti-Fas antibody were harvested. The plasmid DNA contained in each cell population was extracted.

5. The anti-sense cDNA inserts contained in these plasmid DNA samples were preferentially amplified through the use of PCR (see details below).

6. The pools of anti-sense cDNA fragments that were derived from cells after treatment were subtracted from those before treatment (see details below). This generated a set of cDNA fragments that were present in cells before treatment but were absent after treatment. It is likely that expression of some of these fragments leads to the inactivation of genes which causes cells to become super-sensitive to anti-Fas antibody treatment. These super-sensitive cells are killed at a lower dose of anti-Fas antibody or more rapidly than the majority of cells. These cells are therefore lost from the treated cultures but are present in the untreated population. Likewise, the AS-fragment harboring-plasmids inducing this super-sensitivity are present in the cells before treatment but are absent from the cell sample taken after treatment. Thus, these fragments are identified during the subtraction.

7. The cDNA fragments generated by the subtraction were cloned into the original expression vector. Appropriate restriction enzyme sites were generated or maintained during the subtraction procedure so that the recloned construct is exactly identical to the construct in the originally transfected cells. The sequence of the isolated cDNA fragments was determined.

8. The anti-sense expression plasmids containing the cDNA inserts that were identified in the subtraction method were individually re-transfected into HeLa cells and the transfectant cells were assayed for sensitivity to the activating anti-Fas antibody treatment.

### Specific Materials and Methods

HeLa cells were transfected with anti-sense cDNA library cloned in the episomal vector, anti-sense expression vector pTKO-1. This is the same library described in Deiss and Kimchi: A genetic tool used to identify thioredoxin as a mediator of a growth inhibitory signal. Science 1991 Apr. 5; 252(5002):117-20. One million cells plated in a 100 mm dish were transfected with 15 μg of DNA containing the anti-sense cDNA library, by using the Superfect reagent (Qiagen, Santa Clarita, Calif.) as suggested by the manufacturer. Two days following transfection, cells were treated with Hygromycin B (200 μg/ml) (Calbiochem-Novabiochem Corporation, La Jolla, Calif.). Following two weeks of selection, the entire population of cells was resistant to Hygromycin B.

These cells were plated in triplicate at a density of 2.5×10⁶ cells per 150 mm dish in the absence of Hygromycin B. One plate was treated with anti-Fas antibody at 10 ng/ml (clone CHI-11 Kamiya Biomedical Company, Seattle, Wash.) for five days, the second plate was treated with 100 ng/ml of anti-as antibody for 24 hours and the third plate was untreated for 24 hours. Following the treatments, the cells were harvested by washing twice with ice cold PBS (NaCl 8 g/liter; KCl 0.2 g/liter; Na² HPO⁴ 1.44 g/liter; KH² PO⁴ 0.24 g/liter; final pH of solution adjusted to pH 7.4 with HCl) and concentrated by centrifugation (15,000·g for 15 seconds). DNA was extracted by using solutions P1, P2 and P3, from the Qiagen Plasmid Purification Kit (Qiagen, Santa Clarita, Calif.). The cell pellet was resuspended in 200 μl of solution P1 (50 mM Tris-HCl, pH 8.0; 10 mM EDTA; 100 μg/ml RNase A) then mixed with 200 μl of solution P2 (200 mM NaOH, 1% SDS) and incubated five minutes at room temperature. 200 μl of solution P3 (3.0M Potassium Acetate, pH 5.0) were added and incubated two minutes at room temperature, followed by a ten minute centrifugation at 15,000·g. The clear supernatant was mixed with an equal volume of isopropanol and centrifuged at 15,000·g for ten minutes. The precipitated DNA was resuspended in 100 μl of water and stored frozen until use.

For PCR amplification of the cDNA inserts contained in these plasmid DNA preparations, the following reaction was set in a total volume of 100 μl:1 μl of the DNA, 200 μl of dATP, dGTP, dCTP, dTTP, 500 ng of each primer; 10 mM Tris-HCl pH 9.0; 0.1 Triton X-100; 1.0 mM MgCl and 1 unit of Taq DNA polymerase (Gibco/BRL, Gaithersburg, Md.). This reaction was incubated in a Thermocycler 2400 (Perkin-Elmer, Foster City, Calif.) according to the following protocol: First, the reaction was heated to 94° C. for five minutes, then was cycled 25 times using the following three temperatures: 58° C. for one minute, 72° C. for five minutes, 94° C. for one minute. After 25 cycles, the reaction was incubated at 72° C. for seven minutes. This resulted in amplification of the cDNA inserts. The primers were designed such that the end of the cDNA insert that is proximal to the promoter in the pTKO-1 vector is exactly flanked by a HindIII restriction site (this site is present in the vector) and the end of the cDNA that is distal to the promoter in pTKO-1 vector contains a BamHI restriction site. The BamHI site was created by altering a single base in the sequence immediately adjacent to the distal cDNA insert site, by PCR. When the library was generated (see Deiss and Kimchi, above), this site distal to the promoter was generated by the fusion of a BamHI restriction site (derived from the cDNA fragments) and a BglII site (derived from the vector). This fused site is resistant to cleavage by either enzymes, but a single base change restored the cleavage by BamHI. Thus, the amplified cDNA fragments are flanked by a HindIII restriction site on the promoter proximal side and by a BamHI site on the promoter distal side. This allows the exact re-cloning of the fragments into the pTKO-1 expression vector with exact conservation of sequence and orientation. Following the PCR reaction, the mixture was cleaved with BamHI and HindIII (Gibco/BRL, Gaithersburg, Md.) as described by the manufacturer. The digestion products were purified using the Wizard PCR Prep Kit (Promega, Madison, Wis.). This generated cDNA inserts with HindIII and BamHI ends.

These nucleic acid fragments were subjected to subtraction using the PCR-Select Kit (Clontech, Palo Alto, Calif.) according to the instructions of the manufacturer with modifications. The PCR products derived from the untreated samples served as the driver, and two testers were used. The first tester was derived from cells treated with 10 ng/ml anti-Fas antibody and the second tester was derived from cells treated with 100 ng/ml of anti-Fas antibody. The manual supplied by the manufacturer with the kit was followed from the point of ligation of the adapters to the tester (Section IV F3 in the Manual). 0.3 μg of the tester was taken for adapter ligation. The initial hybridization included 0.9 μg of the driver and 0.03 μg of the adapted ligated tester. At the conclusion of the subtraction, a final PCR reaction is done using nested PCR primers. This material contains the cDNA fragments that were present in the untreated sample but absent from the treated samples.

The products of this PCR reaction were re-cloned into the anti-sense expression vector. Re-cloning of the subtracted fragments was accomplished by cleaving the subtracted population with BamHI and HindIII and purifying the cleaved products with the Wizard PCR Prep Kit (Promega Madison, Wis.). The cleaved products were then directly cloned into the pTKO1-DHFR vector between the HindIII and BglII sites. This replaced the DHFR sequences with the cDNA. This is precisely the procedure that was used to generate the anti-sense cDNA expression library. Thus, the fragments that were generated by the subtraction were exactly re-cloned into the original anti-sense expression vector that was used to transfect cells at the beginning of the procedure. The re-cloned constructs exactly duplicate the constructs that were present in the library. The re-cloned constructs were introduced into bacteria and DNA was extracted from the bacteria following conventional methods. These DNA preparations were used as a template for sequencing in order to determine the nucleotide sequence of the isolated cDNA inserts. In addition, plasmids carrying the re-cloned inserts were transfected into HeLa cells to confirm their ability to induced super-sensitization to anti-Fas antibody treatment in HeLa cells.

HeLa cells were transfected with 15 μg of plasmids or control vectors as described for transfection of the original library. The cells were selected for two weeks for resistance to Hygromycin B treatment (200 μg/ml). This selects for cells which contain expression cassettes. One million cells were plated in a 100 mm dish and treated with anti-Fas antibody. Effects of anti-Fas antibody on the transfected cultures were quantified by trypan blue or by FACS analysis.

### Example II

### Validation of the Identified Gene Fragment

### A) The Effect of IDH Antisense Fragment on FAS Induced Apoptosis in HeLa Cells was Tested by Loss of Function (LOF) Assays

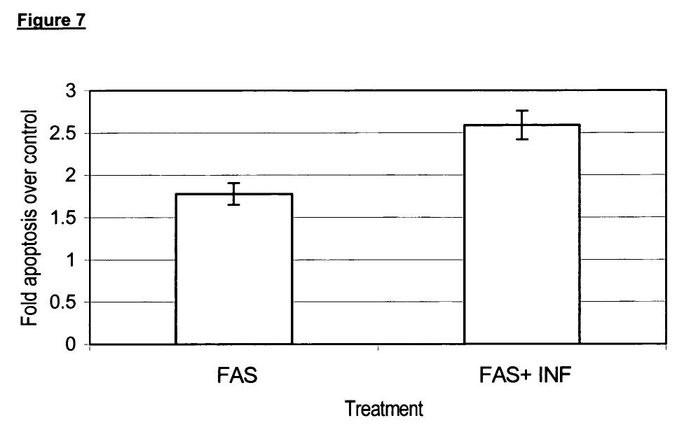

### See FIGS. 7, 8 and 10

HeLa cells were stably transfected with either empty vector (serves as a control) or a vector that contains the IDH anti-sense fragment. After selection by Hygromycin B, pools of HeLa cells expressing the IDH anti-sense were subjected to the FAS killing assay by two sets of experiments:

In the first set of experiments, apoptosis was detected by labeling with AnnexinV-Cy3. (BioVision). During the early stages of apoptosis, cell membranes lose their phospholipid symmetry and expose phosphatidylserine (PS) at the cell surface (Martin, S. J., et al. (1995) J. Exp. Med. 182: 1545-1556). Annexin V, a calcium-dependent phospholipid-binding protein, has a high affinity for PS (Koopman, G., et al. (1994) Blood 84: 1415-1420). The Apoptosis Detection Kits use Annexin V conjugated to various markers or chromophores for convenient detection of apoptotic cells, and many such kits are available.

In the second set of experiments, the cells were pretreated with INF-γ and apoptosis was detected with propidium iodide (which labels dividing cells). The results for the LOF assays are presented in FIG. 7, in the form of fold apoptosis over control. These results demonstrate that cells harboring the IDH antisense fragment were more sensitive to FAS mediated apoptosis than the control cells. Pretreatment with INF-γ increased this effect.

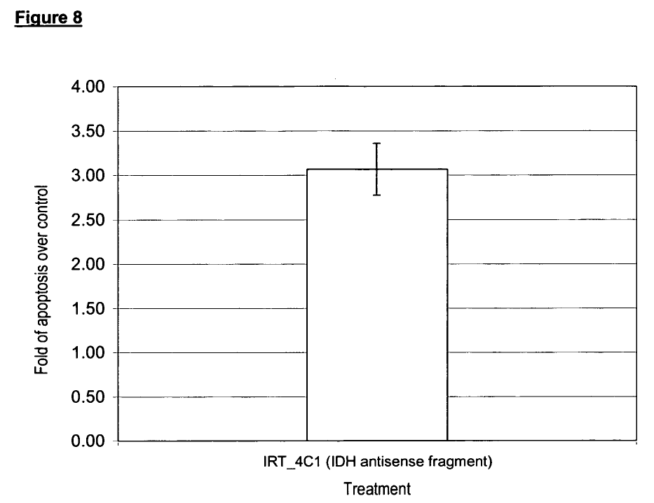

The results of a similar LOF experiment using doxorubicin instead of Fas are depicted in FIG. 8. These experiments were performed according to the following method:

HeLa cells were stably transfected with a vector harboring the sensitizing IDH antisense fragment using the FuGENE 6 transfection reagent (Roche). Selection on hygromicin B (200 ng/ml) was carried out for 10 days at the end of which stable pools were obtained. Stable pools were then plated at a density of 20×10⁴ cell per well of six-wells-plate. After 24 hours cells were treated with doxorubicin (0.5-1 μg/ml) for 24-48 hours. Following doxorubicin treatment, cells were harvested and the level of apoptosis was measured by labeling with annexin-FITC (to circumvent the endogenous red fluorescence of doxorubicin) (BioVision). The fluorescent signal was detected by using a cell sorter (FACSCalibur, Becton Dickinson). Each experiment was done in quadruplicates and two wells were collected into one tube for measurement. The results were calculated using the WinMDI software.

FIG. 8 presents the results in the form of fold of apoptosis (calculated from mean of annexin V signal from positive cells) over control (empty vector harboring cells). These results demonstrate that cells harboring the IDH antisense fragment were more sensitive to Doxorubicin mediated apoptosis than the control cells.

### B) The Effect of IDH siRNA on Doxorubicin Induced Apoptosis in HeLa Cells was Tested by a Loss of Function (LOF) Assay

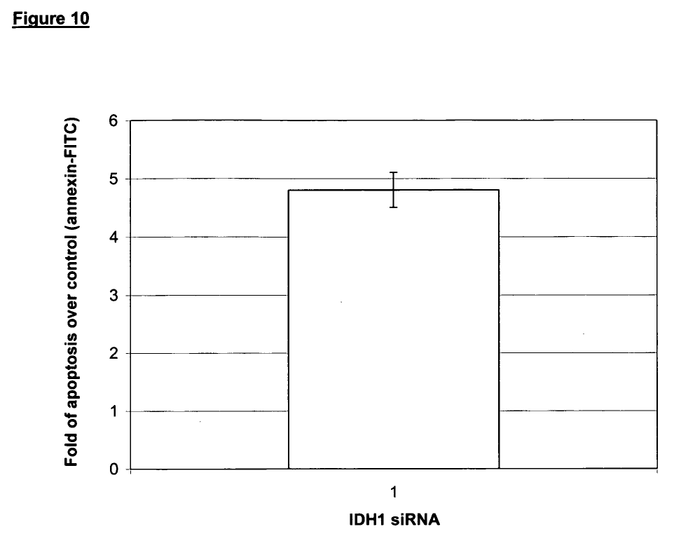

### See FIG. 10

HeLa cells were transiently transfected with an IDH siRNA (5′ AAATCGTGATGCCACCAACGAC′3, synthesized by DHARMACON Research Inc. 1376 Miners Drive #101 Lafayette, Colo. 80026). 48 hours after transfection, cells were treated with doxorubicin (500 ng/ml for 16 h). Apoptosis was measured using an Annexin-V-FITC kit (see above). The results are presented as fold of apoptosis in IDH siRNA transfected cells over apoptosis in LUC transfected cells (control). These results demonstrate that IDH siRNA sensitizes cells to doxorubicin mediated apoptosis.

### C) The Effect of IDH on FAS Induced Apoptosis in HeLa Cells was Tested by Gain of Function (GOF) Assays

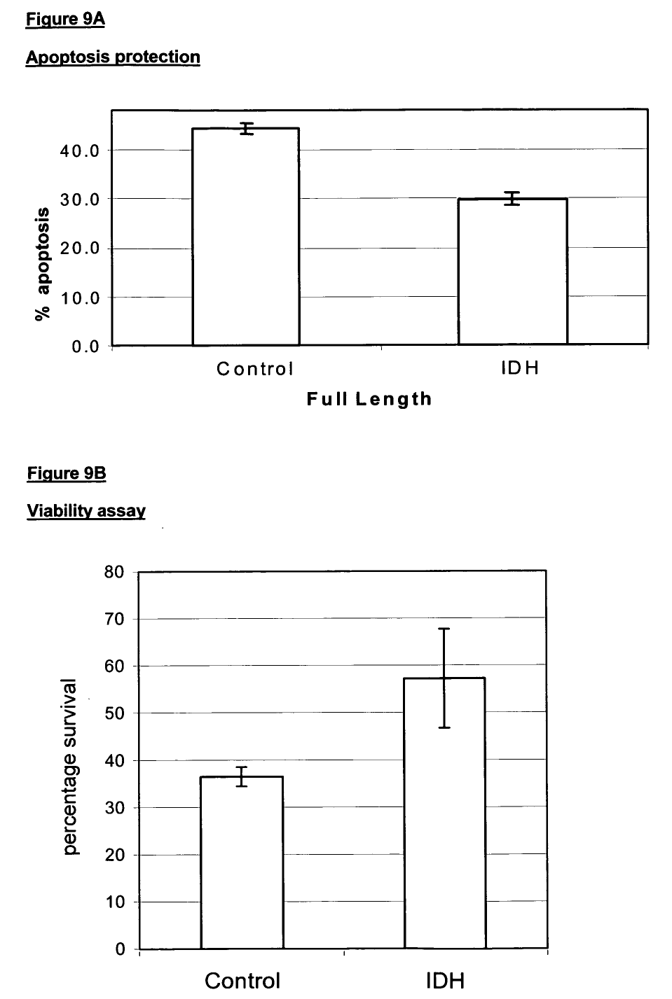

### See FIG. 9

The GOF validation assay was conducted according to the following method:

HeLa cells were transiently transfected with an expression vector harboring the IDH full ORF. The empty vector was used as a negative control. The expression of IDH in the transfected cells was monitored by western blot analysis, using an anti-IDH specific antibody. Four hours after transfection, the cells were treated with anti FAS (CH-11, 500 ng/ml) for 48-72 hours. Apoptosis was detected using annexin V Cy3 and analyzed by FACS. The results were calculated as percentage of apoptosis.

FIG. 9A represents a GOF experiment performed using transient transfection and an apoptosis assay as a readout system. HeLa cells were transiently transfected with pBABE-puro vector harboring the IDH full length ORF or empty vector (control). Four hours after recovery from transfection, cells were treated with anti FAS (CH-11, 500 ng/ml) for 48-72 hours. Apoptosis was detected using annexin V Cy3 and analyzed by FACS. The y axis represents percentage of apoptosis (annexin V positive signal). These results demonstrate that the full length IDH gene protects cells from FAS mediated apoptosis as compared to control cells.

FIG. 9B represents a GOF experiment performed using stable transfection and a viability assay as a readout system. HeLa cells were stably transfected with pBABE-puro vector harboring the IDH full length ORF or empty vector (control). Stable pools were treated with anti FAS (500 ng/ml) for 17-24 hours as described above. The number of viable (i.e.: trypan blue excluding) cells that remained attached to the plate following rinsing with PBS was determined. The y axis represents the percentage of surviving cells (trypan blue negative signal). These results demonstrate that upon treatment with Fas, cells harboring the full length IDH gene have increased viability as compared to control cells.

### Example III

### Administration of Compounds

The compound of the present invention e.g. the inhibitor of the IDH gene or gene product may be administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art.

The compound of the present invention may be administered in various ways. It should be noted that it may be administered as the compound per se or as a pharmaceutically acceptable salt, and may be administered alone or as an active ingredient in combination with pharmaceutically acceptable excipients such as carriers, diluents, adjuvants and vehicles. The compounds may be administered orally, subcutaneously or parenterally including intravenous, intra-arterial, intramuscular, intraperitoneally, and intranasal administration as well as intrathecal and infusion techniques. Implants of the compounds are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

It is noted that humans are treated generally longer than the mice or other experimental animals exemplified herein which treatment has a length proportional to the length of the disease process and drug effectiveness.

The doses may be single doses or multiple doses over a period of several days, but single doses are preferred. The treatment generally has a length proportional to the length of the disease process and drug effectiveness and the patient species being treated.

When administering the compound of the present invention parenterally, it is generally formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier may be a solvent or dispersing medium containing, for example, water, ethanol, polyol (for example, glycerol, propylene glycol, liquid polyethylene glycol, and the like), suitable mixtures thereof, and vegetable oils.

Proper fluidity may be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, may be added. Prevention of the action of microorganisms may be ensured by various antibacterial and antifungal agents, for example, parabens, chlorobutanol, phenol, sorbic acid, and the like. In many cases, it is desirable to include isotonic agents, for example, sugars, sodium chloride, and the like. Prolonged absorption of the injectable pharmaceutical form may be brought about by the use of agents delaying absorption, for example, aluminum monostearate and gelatin. According to the present invention, however, any vehicle, diluent, or additive used have to be compatible with the compounds.

Sterile injectable solutions may be prepared by incorporating the compounds utilized in practicing the present invention in the required amount of the appropriate solvent with various of the other ingredients, as desired.

A pharmacological formulation of the present invention may be administered to the patient in an injectable formulation containing any compatible carrier, such as various vehicle, adjuvants, additives, and diluents; or the compounds utilized in the present invention may be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

A pharmacological formulation of the compound utilized in the present invention may be administered orally to the patient. Conventional methods such as administering the compounds in tablets, suspensions, solutions, emulsions, capsules, powders, syrups and the like are usable. Known techniques which deliver it orally or intravenously and retain the biological activity are preferred. In one embodiment, the compound of the present invention may be administered initially by intravenous injection to bring blood levels to a suitable level. The patient's levels are then maintained by an oral dosage form, although other forms of administration, dependent upon the patient's condition and as indicated above, may be used. The quantity to be administered vary for the patient being treated and vary from about 100 ng/kg of body weight to 100 mg/kg of body weight per day and preferably are from 10 μg/kg to 10 mg/kg per day.

### Example IV

### Screening Assays

The IDH gene may be used in a screening assay for identifying and isolating compounds which inhibit or stimulate apoptosis, and in particular, Fas-induced and drug-induced apoptosis. The compounds to be screened comprise inter alia substances such as small chemical molecules, antibodies, antisense oligonucleotides, antisense DNA or RNA molecules, polypeptides and dominant negatives, and expression vectors. (A synthetic antisense oligonucleotide drug can inhibit translation of mRNA encoding the gene product of a Fas pathway gene.) Small chemical molecules generally have a molecular weight of less than 2000 daltons, more preferably less than 1000 daltons.

Many types of screening assays are known to those of ordinary skill in the art. The specific assay which is chosen depends to a great extent on the activity of the candidate gene or the polypeptide expressed thereby. Thus, if it is known that the expression product of a candidate gene has enzymatic activity, then an assay which is based on inhibition (or stimulation) of the enzymatic activity can be used. This is the case with IDH. If the candidate polypeptide is known to bind to a ligand or other interactor, then the assay can be based on the inhibition of such binding or interaction. When the candidate gene is a known gene, then many of its properties can also be known, and these can be used to determine the best screening assay. If the candidate gene is novel, then some analysis and/or experimentation is appropriate in order to determine the best assay to be used to find inhibitors of the activity of that candidate gene. The analysis can involve a sequence analysis to find domains in the sequence which shed light on its activity.

As is well known in the art, the screening assays can be cell-based or non-cell-based. The cell-based assay is performed using eukaryotic cells such as HeLa cells, and such cell-based systems are particularly relevant in order to directly measure the activity of candidate genes which are anti-apoptotic functional genes, i.e., expression of the gene prevents apoptosis or otherwise prevents cell death in target cells, such as the IDH gene. One way of running such a cell-based assay uses tetracycline-inducible (Tet-inducible) gene expression. Tet-inducible gene expression is well known in the art; see for example, Hofmann et al, 1996, Proc Natl Acad Sci 93(11):5185-5190.

Tet-inducible retroviruses have been designed incorporating the Self-inactivating (SIN) feature of a 3′ Ltr enhancer/promoter retroviral deletion mutant. Expression of this vector in cells is virtually undetectable in the presence of tetracycline or other active analogs. However, in the absence of Tet, expression is turned on to maximum within 48 hours after induction, with uniform increased expression of the whole population of cells that harbor the inducible retrovirus, thus indicating that expression is regulated uniformly within the infected cell population.

When dealing with candidate genes having anti-apoptotic function, such as the IDH gene, Tet-inducible expression prevents apoptosis in target cells. One can screen for chemical compounds able to rescue the cells from the gene-triggered inhibition of apoptosis.

If the gene product of the candidate gene phosphorylates with a specific target protein, a specific reporter gene construct can be designed such that phosphorylation of this reporter gene product causes its activation, which can be followed by a color reaction. The candidate gene can be specifically induced, using the Tet-inducible system discussed above, and a comparison of induced versus non-induced genes provides a measure of reporter gene activation.

In a similar indirect assay, a reporter system can be designed that responds to changes in protein-protein interaction of the candidate protein. If the reporter responds to actual interaction with the candidate protein, a color reaction occurs. One can also measure inhibition or stimulation of reporter gene activity by modulation of its expression levels via the specific candidate promoter or other regulatory elements. A specific promoter or regulatory element controlling the activity of a candidate gene is defined by methods well known in the art. A reporter gene is constructed which is controlled by the specific candidate gene promoter or regulatory elements. The DNA containing the specific promoter or regulatory agent is actually linked to the gene encoding the reporter. Reporter activity depends on specific activation of the promoter or regulatory element. Thus, inhibition or stimulation of the reporter is a direct assay of stimulation/inhibition of the reporter gene; see, for example, Komarov et al (1999), Science vol 285, 1733-7 and Storz et al (1999) Analytical Biochemistry, 276, 97-104.

Various non-cell-based screening assays are also well within the skill of those of ordinary skill in the art. For example, if enzymatic activity is to be measured, such as if the candidate protein has a kinase activity, the target protein can be defined and specific phosphorylation of the target can be followed. The assay can involve either inhibition of target phosphorylation or stimulation of target phosphorylation, both types of assay being well known in the art; for example see Mohney et al (1998) J. Neuroscience 18, 5285 and Tang et al (1997) J. Clin. Invest. 100, 1180 for measurement of kinase activity. *Although this is not relevant in cases where there is no known enzymatic activity, there is a possibility that non enzyme proteins interact with an enzyme and regulate its enzymatic activity through protein-protein interaction.

One can also measure in vitro interaction of a candidate polypeptide with interactors. In this screen, the candidate polypeptide is immobilized on beads. An interactor, such as a receptor ligand, is radioactively labeled and added. When it binds to the candidate polypeptide on the bead, the amount of radioactivity carried on the beads (due to interaction with the candidate polypeptide) can be measured. The assay indicates inhibition of the interaction by measuring the amount of radioactivity on the bead.

Any of the screening assays, according to the present invention, can include a step of identifying and obtaining the chemical compound (as described above) which tests positive in the assay and can also include the further step of producing as a medicament that which has been so identified. It is considered that medicaments comprising such compounds, or chemical analogs or homologs thereof, are part of the present invention. The use of any such compounds identified for inhibition or stimulation of apoptosis, is also considered to be part of the present invention.

Specific In-Vitro Bioassay for Screening for Small Molecules which Inhibit IDH

IDH catalyzes the following enzymatic reaction:

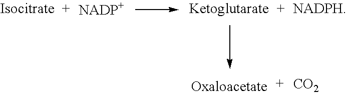

For the purposes of the bioassay, NADPH produced in the enzymatic reaction is reacted with the tetrazolium salt WST-1 according to the following scheme:

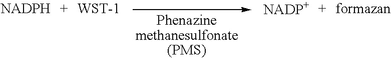

PMS serves to facilitate the electron transfer. The soluble colored formazan product formed in this reaction possesses strong absorption at 450 nm. An excellent linear correlation between the NADPH produced and the amount of formazan formed by NADPH reduction by WST-1 was observed. In the absence of NADPH, minimal spontaneous WST-1 reduction was observed.

### Materials:

Dₛ-threo Isocitric acid, potassium salt at a stock concentration of 1.15 mM was purchased from Fluka; a WST-1 kit was purchased from Roche. β-NADP sodium salt at a stock concentration of 4.6 mM, Maltose, Tris, MgCl₂, Glycerol, β-mercaptoethanol and NaCl were purchased from Sigma.

### Stock Solutions and Reagents:

### Enzyme Storage Buffer:

Tris-HCl—100 mM, pH 8.0, glycerol—10%, NaCl—150 mM, β-mercaptoethanol—2 mM, maltose—10 mM, was prepared fresh just prior to rec. protein purification.

### Assay Buffer (2×):

Tris-HCl—0.4M, pH 8.0, MgCl₂—2 mM.

### NADP Stock Solution (4.6 mM) Preparation and Storage:

3.5 mg β-NADP sodium salt were dissolved in 1 ml DDW.

### Isocitrate Stock Solution (1.15 mM) Preparation and Storage:

2.6 mg (+)-potassium Dₛ-threo-isocitrate were dissolved in 10 ml DDW.

### IDH1 Stock and Working Solutions:

A. Purified IDH1-1.55 munits/μl in enzyme storage buffer (prepared according to the procedure described below). Stored at −80° C.
B. Diluted stock—2 μl of enzyme stock freshly diluted in 88 μl of cold enzyme storage buffer to a working concentration of 35 μunit/μl.
C. Enzyme working solution—0.2 μl IDH enzyme freshly diluted into 24.8 μl 2× reaction buffer (amounts per well).

### Assay Mix (Per Well):

1 μl NADP stock solution (4.6 mM), 3.5 μl isocitrate stock solution (1.15 mM) and 5 μl WST-1 (kit) were mixed. 5.51 μl DDW was added, to a final volume of 15 μl.

### Chemical Library Dilution:

library chemicals were diluted to 1 mM in DMSO, and further diluted at a ratio of 1:20 in DDW just prior to use to obtain 50 μM working solution in 5% DMSO/DDW.

### Assay Stop Solution (50 mM HCl):

10 μl of 300 mM HCl working solution were added to a 50 μl reaction mixture to a final conc. of 50 mM.

### 384-Well Plates:

Greiner. Cat no. 781182

### Assay Procedure:

### Final Reagent Concentrations:

| | Working solutions | | Vol. per well | | |
|---|---|---|---|---|---|
| Compound | (conc.) | | (μl) | Final concentration | |
| _{B}-NADP^{↓} | 4.6 | mM | 1.00 | 92 | μM |
| D_{S}-threo Isocitric acid | 1.15 | mM | 3.50 | 81 | μM |
| 2X reaction buffer | 0.4 | M Tris-HCl, | 25 | 0.2 | M Tris-HCl, |
| | 2 | mM MgCl₂ | | 1 | mM MgCl₂ |
| Enzyme storage buffer | | | | 0.04% | glycerol |
| | | | | 0.6 | mM NaCl |
| | | | | 8 | μM β-mercaptoethanol |
| | | | | 40 | μM maltose |
| IDH1 | 35 | μunit/pμl | | 7 | μunits/well |
| WST-1 | Kit | | 5 | 10% | of stock |
| DDW | | | 5.5 | | |
| HCl (stop) | 300 | mM | 10 | 50 | mM |

### Procedure:

15 μl of the assay mix were dispensed per well (384 well plate).
10 μl of library chemical were added (50 μM in 5% DMSO/DDW) to each well. (Final concentration: 10 μM library chemical; 1% DMSO).
25 μl of enzyme working solution in 2× reaction buffer were added to each well.
The reaction was incubated for 60 min at room temperature.
10 μl HCl stop solution (50 mM final) were added to each well.
The reaction was incubated for up to an additional 30 min at room temperature.
The reaction absorbance was read at 450 nm for 0.1 second on a Victor Fluorescence reader.

### Characteristic Assay Parameters:

### Z′ Score:

IDH1: Z′=0.5, SD±14%.

### Biochemical Parameters:

IDH1: Kₘ (Isocitrate)=19 μM; Kₘ (NADP⁺)=23 μM.

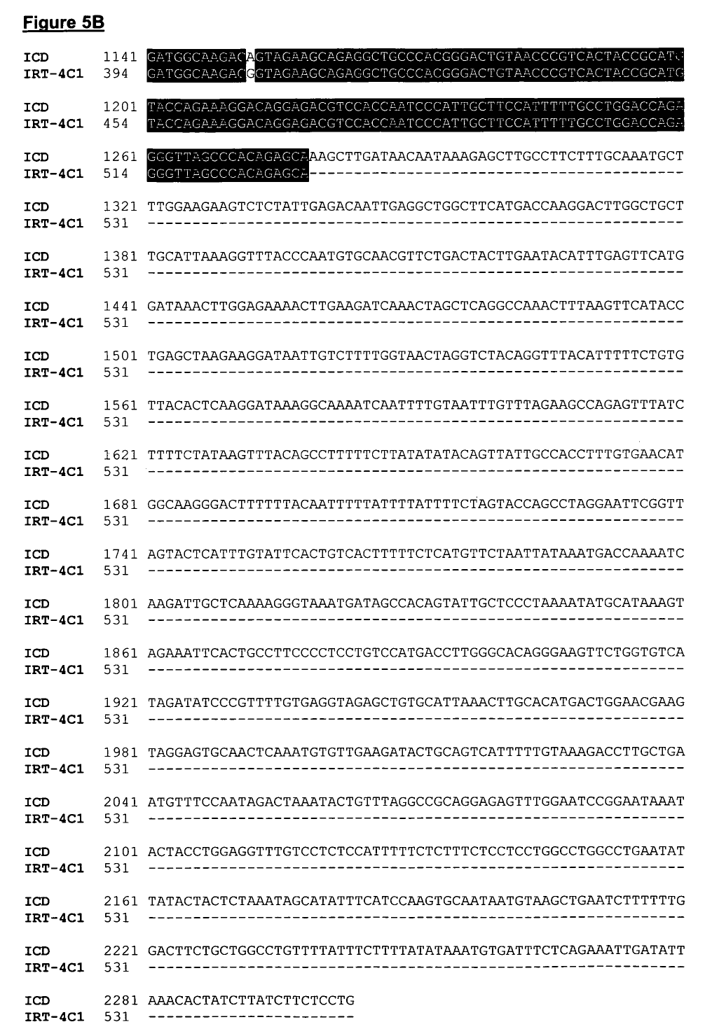

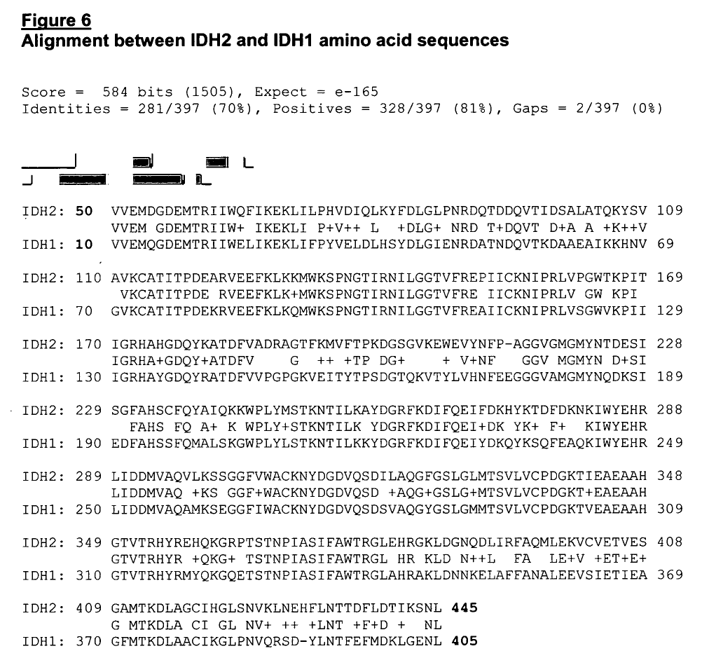

IDH2 and IDH1 are highly homologous (70% polypeptide identity, 81% polypeptide homology—see FIG. 6). As noted herein, the embodiments of the present invention may be practiced with IDH2, IDH1 or both in conjunction. Therefore, the present screening system may be practiced with both IDH1 and/or IDH2 (i.e., separately or together). Should specific inhibitors be desired that act on one but not the other, the screen can be performed in duplicate with both enzymes, and the “hits” obtained for the “undesired” enzyme (as the case may be) can be subtracted from the “hits” obtained for the desired enzyme, thereby generating a group of “hits” that are specific for the “desired” enzyme. A similar procedure can be performed to avoid inhibitors for IDH3 (although it is not likely that the above screening systems will identify IDH3 inhibitors, due to low homology).

Cell-Based Secondary Bioassay for Validation of Molecules which Inhibit IDH: Potentiation of Chemotherapy with JDH Inhibitors

In this assay, an IDH inhibitor is administered to mammalian tumor or cancer cell lines, such as human cells derived from breast or colon cancers. The IDH inhibitor is administered to the cells in conjunction with a cancer treatment, such as a chemotherapeutic drug (see above). The viability of the cells as a result of this dual treatment is then examined, and subsequently compared to the viability of control cells (i.e., cells treated with the cancer treatment without the IDH inhibitor). A decreased viability of the dually treated cells as compared to the viability of the control cells validates the inhibitory activity of the inhibitor, and indicates the ability of the inhibitor to potentiate the cells to the cancer treatment.

Examples of viability assays that can be used with this bioassay include Annexin V stain (for apoptosis), and alamar blue or neutral red stains (for life/death).

### Example V

### Preparation of Polypeptides

Polypeptides may be produced via several methods, for example:

### 1) Synthetically;

Synthetic polypeptides can be made using a commercially available machine, using the known sequence of the desired polypeptide.

### 2) Recombinant Methods:

A preferred method of making polypeptides is to clone a polynucleotide comprising the cDNA of the gene of the desired polypeptide into an expression vector and culture the cell harboring the vector so as to express the encoded polypeptide, and then purify the resulting polypeptide, all performed using methods known in the art as described in, for example, Marshak et al., “Strategies for Protein Purification and Characterization. A laboratory course manual.” CSHL Press (1996). (in addition, see Bibl Haematol. 1965; 23:1165-74 Appl Microbiol. 1967 July; 15(4):851-6; Can J. Biochem. 1968 May; 46(5):441-4; Biochemistry. 1968 July; 7(7):2574-80; Arch Biochem Biophys. 1968 Sep. 10; 126(3):746-72; Biochem Biophys Res Commun. 1970 Feb. 20; 38(4):825-30)).

The expression vector can include a promoter for controlling transcription of the heterologous material and can be either a constitutive or inducible promoter to allow selective transcription. Enhancers that can be required to obtain necessary transcription levels can optionally be included. The expression vehicle can also include a selection gene.

Vectors can be introduced into cells or tissues by any one of a variety of methods known within the art. Such methods can be found generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Vega et al., Gene Targeting, CRC Press, Ann Arbor, Mich. (1995), Vectors: A Survey of Molecular Cloning Vectors and Their Uses, Butterworths, Boston Mass. (1988) and Gilboa et al. (1986).

3) Purification from Natural Sources:

Desired polypeptides can be purified from natural sources (such as tissues) using many methods known to one of ordinary skill in the art, such as for example: immuno-precipitation, or matrix-bound affinity chromatography with any molecule known to bind the desired polypeptide.

Protein purification is practiced as is known in the art as described in, for example, Marshak et al., “Strategies for Protein Purification and Characterization. A laboratory course manual.” CSHL Press (1996).

The IDH1 and/or IDH2 Used in the Screening Systems of the Present Invention was Prepared According to the Following:

3 liters of BL-21* bacteria cells harboring a vector containing the IDH1 or IDH2 polypeptide encoding sequence under the control of a LAC promoter were cultured in 37° C., with 0.25 mM IPTG; induction was performed for 3 h. Total proteins were then extracted at 1:20 (Vₑₓₜ/V_{culture}) using an extraction buffer (EB) containing: 100 mM Tris, pH 8.0, 150 mM NaCl, 10% glycerol, 2 mM α-mercoptoethanol and EDTA-Free Cocktail Protease Inhibitors (Roche), and purified according to the following method: 150 ml of the extract was loaded on 10 ml NINTA beads, in 4° C.; the column was washed with 1 M NaCl in EB, and subsequently with 40 mM imidazole in EB. Elution of the IDH1 or IDH2 polypeptide was done with 200 mM imidazole in EB. Optionally for MBP tagged proteins (see below), the sample was then loaded (0.5 ml/min) on a 4 ml Amilose Resin Column, and elution was performed with 10 mM maltose in EB. The final volume of the eluted protein was 5 ml. At this point the protein was ready for use in the activity assay; optionally, an additional step was performed in which 2.5 ml of the protein sample was loaded on a PD10 (G-25 column) for desalting of the maltose. The IDH prepared according to the above was optionally histidine and/or maltose binding protein (MBP) tagged.

The IDH1 protein prepared according to the above was found to be at least 99% pure, and to have a specific activity of 3.87 U/mg protein. Under identical conditions, the IDH2 protein prepared according to the above exhibited a specific activity of 3.24 U/mg protein. (Unit definition: one unit of IDH will catalyze the isocitrate-dependent reduction of 1 μmol NADP to NADPH in 1 min at 25° C. under standard activity conditions. Standard activity conditions: 1 μg IDH, 100 mM Tris-HCl pH 8.0, 150 mM NaCl, 2 mM β-mercapto-ethanol, 0.14% glycerol, 6 mM MgCl2, 3 mM D-L isocitrate, 1 mM NADP. Components were incubated at 25° C. and the final reaction volume was 200 μl).

### Example VI

### Preparation of Polynucleotides

The polynucleotides of the subject invention can be constructed by using a commercially available DNA synthesizing machine; overlapping pairs of chemically synthesized fragments of the desired gene can be ligated using methods well known in the art (e.g., see U.S. Pat. No. 6,121,426).

Another means of isolating a polynucleotide is to obtain a natural or artificially designed DNA fragment based on that sequence. This DNA fragment is labeled by means of suitable labeling systems which are well known to those of skill in the art; see, e.g., Davis et al. (1986). The fragment is then used as a probe to screen a lambda phage cDNA library or a plasmid cDNA library using methods well known in the art; see, generally, Sambrook et al., Molecular Cloning: A Laboratory Manual, Cold Spring Harbor Laboratory Press, New York (1989), in Ausubel et al., Current Protocols in Molecular Biology, John Wiley and Sons, Baltimore, Md. (1989),

Colonies can be identified which contain clones related to the cDNA probe and these clones can be purified by known methods. The ends of the newly purified clones are then sequenced to identify full-length sequences. Complete sequencing of full-length clones is performed by enzymatic digestion or primer walking. A similar screening and clone selection approach can be applied to clones from a genomic DNA library.

The polynucleotide sequences disclosed herein may be used for the purpose of obtaining or preparing the polynucleotides of the present invention.

### Example VII

### Preparation of Anti-IDH Antibodies

Antibodies which bind to the IDH polypeptide may be prepared using an intact polypeptide or fragments containing smaller polypeptides as the immunizing antigen. For example, it may be desirable to produce antibodies that specifically bind to the N- or C-terminal or any other suitable domains of the IDH polypeptide. The polypeptide used to immunize an animal can be derived from translated cDNA or chemical synthesis which can be conjugated to a carrier protein, if desired. Such commonly used carriers which are chemically coupled to the polypeptide include keyhole limpet hemocyanin (KLH), thyroglobulin, bovine serum albumin (BSA) and tetanus toxoid. The coupled polypeptide is then used to immunize the animal.

If desired, polyclonal or monoclonal antibodies can be further purified, for example by binding to and elution from a matrix to which the polypeptide or a peptide to which the antibodies were raised is bound. Those skilled in the art know various techniques common in immunology for purification and/or concentration of polyclonal as well as monoclonal antibodies (Coligan et al, Unit 9, Current Protocols in Immunology, Wiley Interscience, 1994).

Methods for making antibodies of all types, including fragments, are known in the art (See for example, Harlow and Lane, Antibodies: A Laboratory Manual, Cold Spring Harbor Laboratory, New York (1988)). Methods of immunization, including all necessary steps of preparing the immunogen in a suitable adjuvant, determining antibody binding, isolation of antibodies, methods for obtaining monoclonal antibodies, and humanization of monoclonal antibodies are all known to the skilled artisan

The antibodies may be humanized antibodies or human antibodies. Antibodies can be humanized using a variety of techniques known in the art including CDR-grafting (EP239,400: PCT publication WO 91/09967; U.S. Pat. Nos. 5,225,539; 5,530,101; and 5,585,089, veneering or resurfacing (EP 592,106; EP 519,596; Padlan, Molecular Immunology 28(4/5):489-498 (1991); Studnicka et al., Protein Engineering 7(6):805-814 (1994); Roguska et al., PNAS 91:969-973 (1994)), and chain shuffling (U.S. Pat. No. 5,565,332).

The monoclonal antibodies as defined include antibodies derived from one species (such as murine, rabbit, goat, rat, human, etc.) as well as antibodies derived from two (or more) species, such as chimeric and humanized antibodies.

Completely human antibodies are particularly desirable for therapeutic treatment of human patients. Human antibodies can be made by a variety of methods known in the art including phage display methods using antibody libraries derived from human immunoglobulin sequences. See also U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Additional information regarding all types of antibodies, including humanized antibodies, human antibodies and antibody fragments can be found in WO 01/05998, which is incorporated herein by reference in its entirety.

1. A method for treating a subject having an apoptosis-related disease comprising administering to said subject a therapeutically effective amount of an inhibitor of an isocitrate dehydrogenase (IDH) polypeptide, wherein the IDH polypeptide has an amino acid sequence as set forth in SEQ ID NO:2 or SEQ ID NO:4 or has a sequence which is modified therefrom while retaining the biological properties of IDH, in a dosage sufficient to inhibit expression of the IDH polypeptide so as to thereby treat the subject.

2. A method according to claim 1, wherein the inhibitor is administered in conjunction with a chemotherapeutic agent.

3. (canceled)

4. A method according to claim 1, wherein the inhibitor is a chemical molecule selected from the group consisting of 2-(4-bromo-2,3-dioxobutylthio)-1, N6-ethenoadenosine 2′,5′-bisphosphate, NADP oxoglutatrate, o-(carboxymethyl)oxalohydroxamate, oxalylglycine, 3-bromo-2-ketoglutarate, beta-mercapto-alpha-ketoglutarate, beta-methylmercapto-alpha-ketoglutarate, beta-methylmercapto-alpha-hydroxyglutarate, adriamycin and alpha-methylisocitrate.

5. A method according to claim 1, wherein the inhibitor is an AS fragment comprising consecutive nucleotides having a sequence as set forth in SEQ ID NO:5.

6. A method according to claim 1, wherein the apoptosis-related disease is a cancer.

7. A method for potentiating a chemotherapeutic treatment of a subject having an apoptosis-related disease comprising administering to said subject a therapeutically effective amount of an inhibitor of a human isocitrate dehydrogenase (IDH) polypeptide in conjunction with a chemotherapeutic agent so as to thereby treat the subject; wherein the IDH polypeptide has an amino acid sequence as set forth in SEQ ID NO:2 or SEQ ID NO:4 or has a sequence which is modified therefrom while retaining the biological properties of IDH.

8. (canceled)

9. A method according to claim 7, wherein the inhibitor is a chemical molecule selected from the group consisting of 2-(4-bromo-2,3-dioxobutylthio)-1, N6-ethenoadenosine 2′,5′-bisphosphate, NADP oxoglutatrate, o-(carboxymethyl)oxalohydroxamate, oxalylglycine, 3-bromo-2-ketoglutarate, beta-mercapto-alpha-ketoglutarate, beta-methylmercapto-alpha-ketoglutarate, beta-methylmercapto-alpha-hydroxyglutarate, adriamycin and alpha-methylisocitrate.

10. A method according to claim 7, wherein the inhibitor is an AS fragment comprising consecutive nucleotides having the a sequence as set forth in SEQ ID NO:5.

11. A method according to claim 7, wherein the apoptosis-related disease is a cancer.

12. An antisense oligonucleotide capable of inhibiting the expression of the IDH polypeptide, having a sequence as set forth in SEQ ID NO:5.

13. An expression vector comprising a nucleic acid encoding the antisense oligonucleotide of claim 12.

14.-24. (canceled)

25. A method of sensitizing a cancerous cell to apoptosis comprising the step of contacting the cell with an inhibitor of isocitrate dehydrogenase (IDH).

26. The method according to claim 25, wherein the inhibitor of isocitrate dehydrogenase (IDH) comprises an siRNA.

27. The method according to claim 25, wherein the inhibitor of isocitrate dehydrogenase (IDH) comprises an antisense oligonucleotide.

28. The method according to claim 25, wherein the apoptosis is Fas-induced or chemo-induced apoptosis.


SEQUENCE LISTING (6 sequences)

SEQ ID NO: 1 - DNA, 2301 bp, Homo sapiens
  CDS (247)..(1491)
ggcggcgaagcgggggcacgccctcgcacacgcagagataaattgtgctcccatgacctttatttggaaagtgcctgcgggcctaaaattggcctttgtcccaccgagtacactcagcactgtactttaaaccggataaactgggctgtctggcaggcgataaactacattcagttgagtctgcaagactgggaggaactggggtgataagaaatctattcactgtcaaggtttattgaagtcaaaatgtccaaaaaaatcagtggcggttctgtggtagagatgcaaggagatgaaatgacacgaatcatttgggaattgattaaagagaaactcatttttccctacgtggaattggatctacatagctatgatttaggcatagagaatcgtgatgccaccaacgaccaagtcaccaaggatgctgcagaagctataaagaagcataatgttggcgtcaaatgtgccactatcactcctgatgagaagagggttgaggagttcaagttgaaacaaatgtggaaatcaccaaatggcaccatacgaaatattctgggtggcacggtcttcagagaagccattatctgcaaaaatatcccccggcttgtgagtggatgggtaaaacctatcatcataggtcgtcatgcttatggggatcaatacagagcaactgattttgttgttcctgggcctggaaaagtagagataacctacacaccaagtgacggaacccaaaaggtgacatacctggtacataactttgaagaaggtggtggtgttgccatggggatgtataatcaagataagtcaattgaagattttgcacacagttccttccaaatggctctgtctaagggttggcctttgtatctgagcaccaaaaacactattctgaagaaatatgatgggcgttttaaagacatctttcaggagatatatgacaagcagtacaagtcccagtttgaagctcaaaagatctggtatgagcataggctcatcgacgacatggtggcccaagctatgaaatcagagggaggcttcatctgggcctgtaaaaactatgatggtgacgtgcagtcggactctgtggcccaagggtatggctctctcggcatgatgaccagcgtgctggtttgtccagatggcaagacagtagaagcagaggctgcccacgggactgtaacccgtcactaccgcatgtaccagaaaggacaggagacgtccaccaatcccattgcttccatttttgcctggaccagagggttagcccacagagcaaagcttgataacaataaagagcttgccttctttgcaaatgctttggaagaagtctctattgagacaattgaggctggcttcatgaccaaggacttggctgcttgcattaaaggtttacccaatgtgcaacgttctgactacttgaatacatttgagttcatggataaacttggagaaaacttgaagatcaaactagctcaggccaaactttaagttcatacctgagctaagaaggataattgtcttttggtaactaggtctacaggtttacatttttctgtgttacactcaaggataaaggcaaaatcaattttgtaatttgtttagaagccagagtttatcttttctataagtttacagcctttttcttatatatacagttattgccacctttgtgaacatggcaagggacttttttacaatttttattttattttctagtaccagcctaggaattcggttagtactcatttgtattcactgtcactttttctcatgttctaattataaatgaccaaaatcaagattgctcaaaagggtaaatgatagccacagtattgctccctaaaatatgcataaagtagaaattcactgccttcccctcctgtccatgaccttgggcacagggaagttctggtgtcatagatatcccgttttgtgaggtagagctgtgcattaaacttgcacatgactggaacgaagtaggagtgcaactcaaatgtgttgaagatactgcagtcatttttgtaaagaccttgctgaatgtttccaatagactaaatactgtttaggccgcaggagagtttggaatccggaataaatactacctggaggtttgtcctctccatttttctctttctcctcctggcctggcctgaatattatactactctaaatagcatatttcatccaagtgcaataatgtaagctgaatcttttttggacttctgctggcctgttttatttcttttatataaatgtgatttctcagaaattgatattaaacactatcttatcttctcc

SEQ ID NO: 2 - protein, 414 aa, Homo sapiens
MSKKISGGSVVEMQGDEMTRIIWELIKEKLIFPYVELDLHSYDLGIENRDATNDQVTKDAAEAIKKHNVGVKCATITPDEKRVEEFKLKQMWKSPNGTIRNILGGTVFREAIICKNIPRLVSGWVKPIIIGRHAYGDQYRATDFVVPGPGKVEITYTPSDGTQKVTYLVHNFEEGGGVAMGMYNQDKSIEDFAHSSFQMALSKGWPLYLSTKNTILKKYDGRFKDIFQEIYDKQYKSQFEAQKIWYEHRLIDDMVAQAMKSEGGFIWACKNYDGDVQSDSVAQGYGSLGMMTSVLVCPDGKTVEAEAAHGTVTRHYRMYQKGQETSTNPIASIFAWTRGLAHRAKLDNNKELAFFANALEEVSIETIEAGFMTKDLAACIKGLPNVQRSDYLNTFEFMDKLGENLKIKLAQAKL

SEQ ID NO: 3 - DNA, 1740 bp, Homo sapiens
  CDS (87)..(1445)
ccagcgttagcccgcggccaggcagccgggaggagcggcgcgcgctcggacctctcccgccctgctcgttcgctctccagcttgggatggccggctacctgcgggtcgtgcgctcgctctgcagagcctcaggctcgcggccggcctgggcgccggcggccctgacagcccccacctcgcaagagcagccgcggcgccactatgccgacaaaaggatcaaggtggcgaagcccgtggtggagatggatggtgatgagatgacccgtattatctggcagttcatcaaggagaagctcatcctgccccacgtggacatccagctaaagtattttgacctcgggctcccaaaccgtgaccagactgatgaccaggtcaccattgactctgcactggccacccagaagtacagtgtggctgtcaagtgtgccaccatcacccctgatgaggcccgtgtggaagagttcaagctgaagaagatgtggaaaagtcccaatggaactatccggaacatcctgggggggactgtcttccgggagcccatcatctgcaaaaacatcccacgcctagtccctggctggaccaagcccatcaccattggcaggcacgcccatggcgaccagtacaaggccacagactttgtggcagaccgggccggcactttcaaaatggtcttcaccccaaaagatggcagtggtgtcaaggagtgggaagtgtacaacttccccgcaggcggcgtgggcatgggcatgtacaacaccgacgagtccatctcaggttttgcgcacagctgcttccagtatgccatccagaagaaatggccgctgtacatgagcaccaagaacaccatactgaaagcctacgatgggcgtttcaaggacatcttccaggagatctttgacaagcactataagaccgacttcgacaagaataagatctggtatgagcaccggctcattgatgacatggtggctcaggtcctcaagtcttcgggtggctttgtgtgggcctgcaagaactatgacggagatgtgcagtcagacatcctggcccagggctttggctcccttggcctgatgacgtccgtcctggtctgccctgatgggaagacgattgaggctgaggccgctcatgggaccgtcacccgccactatcgggagcaccagaagggccggcccaccagcaccaaccccatcgccagcatctttgcctggacacgtggcctggagcaccgggggaagctggatgggaaccaagacctcatcaggtttgcccagatgctggagaaggtgtgcgtggagacggtggagagtggagccatgaccaaggacctggcgggctgcattcacggcctcagcaatgtgaagctgaacgagcacttcctgaacaccacggacttcctcgacaccatcaagagcaacctggacagagccctgggcaggcagtagggggaggcgccacccatggctgcagtggaggggccagggctgagccggcgggtcctcctgagcgcggcagagggtgagcctcacagcccctctctggaggcctttctaggggatgtttttttataagccagatgtttttaaaagcatatgtgtgtttcccctcatggtgacgtgaggcaggagcagtgcgttttacctcagccagtcagtatgttttgcatactgtaatttatattgcccttggaacacatggtgccatatttagctactaaaaagctcttcacaaaaaaaaaaa

SEQ ID NO: 4 - protein, 452 aa, Homo sapiens
MAGYLRVVRSLCRASGSRPAWAPAALTAPTSQEQPRRHYADKRIKVAKPVVEMDGDEMTRIIWQFIKEKLILPHVDIQLKYFDLGLPNRDQTDDQVTIDSALATQKYSVAVKCATITPDEARVEEFKLKKMWKSPNGTIRNILGGTVFREPIICKNIPRLVPGWTKPITIGRHAHGDQYKATDFVADRAGTFKMVFTPKDGSGVKEWEVYNFPAGGVGMGMYNTDESISGFAHSCFQYAIQKKWPLYMSTKNTILKAYDGRFKDIFQEIFDKHYKTDFDKNKIWYEHRLIDDMVAQVLKSSGGFVWACKNYDGDVQSDILAQGFGSLGLMTSVLVCPDGKTIEAEAAHGTVTRHYREHQKGRPTSTNPIASIFAWTRGLEHRGKLDGNQDLIRFAQMLEKVCVETVESGAMTKDLAGCIHGLSNVKLNEHFLNTTDFLDTIKSNLDRALGRQ

SEQ ID NO: 5 - DNA, 531 bp, Homo sapiens
tgctctgtgggctaaccctctggtccaggcaaaaatggaagcaatgggattggtggacgtctcctgtcctttctggtacatgcggtagtgacgggttacagtcccgtgggcagcctctgcttctaccgtcttgccatctggacaaaccagcacgctggtcatcatgccgagagagccatacccttgggccacagagtccgactgcacgtcaccatcatagtttttacaggcccagatgaagcctccctctgatctcatagctggggccaccatgtcgtcgatgagcctatgctcataccagatcttttgagcttcaaactgggacttgtactgcttgtcatatatctcctgaaagatgtctttaaaacgcccatcatatttcttcagaatggtgtttttggtgctcagatacaaaggccaacccttagacagagccatttggaaggaactgtgtgcaaaatcttcaattgacttatcttgattatacatccccatgacaacaccaccaccttcttcaagttatgtaccagg

SEQ ID NO: 6 - DNA, 21 bp, Homo sapiens
aaucgugaugccaccaacgac